(19) United States
(12) Patent Application Publication
(10) Pub. No.: US 2012/0010196 A1
(43) Pub. Date: Jan. 12, 2012

(54) METHODS OF TREATING NEURODEGENERATIVE DISORDERS AND DISEASES

(76) Inventors: Quingyu QIN, Pomona, CA (US); Xiaoning Bl, Irvine, CA (US)

(21) Appl. No.: 13/079,528

(22) Filed: Apr. 4, 2011

Related U.S. Application Data
(60) Provisional application No. 61/320,508, filed on Apr. 2, 2010.
(60) Provisional application No. 61/320,503, filed on Apr. 2, 2010.

(51) Int. Cl. A61K 31/551; A61P 25/16; A61P 25/28; C12Q 1/02; A61K 31/4409

(52) U.S. Cl. 514/218; 435/29; 514/352

(57) ABSTRACT

This invention is directed to a novel method of treating neurodegenerative disorders and diseases. Another, related aspect of this invention is directed to a screening method of identifying compounds that can be used to treat neurodegenerative disorders and diseases. The foregoing aspects of the invention particularly relate to neurodegenerative disorders and diseases have degeneration of neuronal axons as part of their pathologies. The method of treatment involves administering a pharmaceutical formulation that comprises a compound or mixture of compounds that inhibits one or more intracellular signaling mechanism that regulate axon degeneration or growth cone collapse. The screening method aspect of the invention identifies test compounds that can be used for the treatment or prevention of neurodegenerative disorders based on the test compound's ability to inhibit axon degeneration or growth cone collapse.

### CROSS REFERENCE TO RELATED APPLICATION

This application claims benefit of priority of U.S. Provisional Patent Application Ser. No. 61/320,508 filed on Apr. 2, 2010, and U.S. Provisional Patent Application Ser. No. 61/320,503 filed on Apr. 2, 2010, the entire disclosures of which are incorporated herein by reference.

### BACKGROUND

Axonal degeneration is a common feature of many neurodegenerative diseases, including Alzheimer's disease (AD), amyotrophic lateral sclerosis, Parkinson's disease, and Niemann-Pick type C (NPC) disease. NPC disease is caused by mutations in NPC1 or NPC2 gene, with late endosomal/lysosomal cholesterol accumulation as its characteristic pathologic feature. Although NPC proteins are ubiquitously distributed in the body and regulate intracellular cholesterol trafficking [1], the most prominent pathological feature of the disease is progressive neuronal death, particularly of neurons in cerebellum, cortex, thalamus and brainstem [reviewed in [2]]. Neuronal degeneration as well as other neuropathological features, including abnormal formation of meganeurites (spindle-shaped swelling in the initial segments of axons) and axonal spheroids, and inflammation have been reproduced in murine models of the disease [3,4,5,6].

NPC pathology shares several features with AD pathology, including neurofibrillary tangles, autophagic/lysosomal dysfunction, inflammation, and cholesterol metabolism abnormalities [7,8,9,10]. In some late onset NPC cases, amyloid plaques dependent on ApoE4 genotype are also present in certain parts of the brain [11]. Thus, NPC has often been used as a model system to study AD pathology.

Axonal degeneration together with intraneuronal cholesterol accumulation can be detected as early as postnatal day 9 in mice with mutant Npc1 proteins (npc1−/− mice) [12]. In vitro experiments with sympathetic neurons cultured from npc1−/− mice showed that, in parallel with cholesterol accumulation in late endosomes/lysosomes, cholesterol levels were decreased in the distal portions of axons [13]. Treatment of cultured hippocampal neurons from wild-type mice with the cholesterol transport inhibitor, U18666A, leads to a reduction in cholesterol content in axonal plasma membranes [14]. As inhibition of cholesterol synthesis induces axonal growth impairment [15], these results raise the possibility that cholesterol deficiency in axons may contribute to the axonal abnormalities found in NPC and other neurodegenerative diseases. In addition, defects in vesicle trafficking and abnormal autophagic/lysosomal function reported to be present in npc1−/− mice [7] could also affect axonal growth.

Axonal growth during development and axonal regeneration in adult nervous system depends on the motility of axonal growth cones, which are dynamic, actin-supported extensions of developing axons seeking their synaptic target. The dynamics as well as the directional motility of axonal growth cones are governed by both intrinsic factors and environmental clues. Guirland et al. recently showed that brain-derived neurotrophic factor (BDNF)-induced growth cone attraction was eliminated by membrane cholesterol depletion, and rescued by subsequent cholesterol restoration [16]. Likewise, growth cone repulsion induced by netrin-1 or semaphorin 3A was also disrupted by cholesterol depletion [16], indicating that membrane cholesterol is critically involved in the regulation of growth cone responses to environmental cues.

The tumor suppressor protein p53 also regulates growth cone motility through a transcription-independent mechanism [17]. That mechanism can be triggered by a disruption of cholesterol egress from late endosomes/lysosomes induced by NPC1 deficiency or pharmacological manipulation of intracellular cholesterol transport. More specifically, the disruption of cholesterol transport can result in growth cone collapse that is associated with abnormal activation of p38 mitogenactivated protein kinase (MAPK), which in turn leads to Mdm2-dependent p53 degradation. Loss of p53 leads to increased RhoA protein synthesis followed by Rho kinase activation and growth cone collapse. This pathway plays a critical role in the pathogenesis of axonal diseases.

### SUMMARY OF THE INVENTION

This invention is directed to a novel method of treating neurodegenerative disorders and diseases. Another, related aspect of this invention is directed to a screening method of identifying compounds that can be used to treat neurodegenerative disorders and diseases. More specifically, the foregoing aspects of the invention particularly relate to neurodegenerative disorders and diseases have degeneration of neuronal axons as part of their pathologies. Indeed, axon degeneration is a feature of such neurodegenerative disorders as Alzheimer's disease, amyotrophic lateral sclerosis, Parkinson's disease, and Niemann-Pick type C disease.

Briefly, the method of treatment involves administering a pharmaceutical formulation that comprises a compound or mixture of compounds that inhibits one or more intracellular signaling mechanism that regulate axon degeneration. Generally, the method administers the formulation to a mammal such that the active agent contacts at least one component of a mammal's central nervous system such as a sensory or motor nerve, an autonomic nervous system component, or an enteric nervous system component.

Cell signaling pathways that are typically targeted by the method of treatment are involved in the regulation of actin organization, assembly and contraction. Such pathways are known to be subject to downstream regulation by members of the p38 MAPK family, and their target, the E3 ubiquitan ligase, Mdm2. Activation of the p38 MAPK/Mdm2 pathway directly leads to the proteosomal degradation of the p53 tumor suppressor, which in turn, is thought to result in the activation of ROCK by at least two different mechanisms: 1) via activation by RhoA; and 2) by the loss of a direct interaction between ROCK and p53. Activated ROCK then phosphorylates substrate targets like MLC, MLC phosphatase, and LIMK, which then function to cause actin fiber contraction and growth cone collapse.

In one aspect of the method of treatment, growth cone collapse is reduced by administration of a formulation that comprises a ROCK inhibitor compound. Specifically, the method of treatment includes the ROCK inhibitor trans-4-[(1R)-1-Aminoethyl]-N-4-pyridinylcyclohexanecarboxamide dihydrochloride, which is known commercially as Y-27632, as well as (S)-(+)-2-Methyl-1-[(4-methyl-5-isoquinolinyl)sulfonyl]-hexahydro-1H-1,4-diazepine dihydrochloride, which is known commercially as H 1152. By inhibiting ROCK in the method of treatment, Y-27632 and H 1152 block signaling events that are initiated when p53 degradation occurs as a consequence of p38 MAPK activation.

As stated above, the invention also relates to a method of screening compounds that can be used for the treatment or prevention of neurodegenerative disorders that are associated with axon degeneration. Briefly, the method of screening selects compounds that have an ability to inhibit a cell signaling pathway that regulates axon degradation.

Another feature of the screening method is that it relies on primary mammalian neuron cultures for testing compounds. A general feature of the screening method is to administer an agent that induces primary neurons to undergo growth cone collapse or neuron degeneration. The method of screening then involves testing the ability of compounds to inhibit neuronal growth cone collapse and degeneration. Test compounds that significantly inhibit growth cone collapse or axon degeneration become candidate drugs for treating neurodegenerative disorders and diseases as explained above.

As an alternative to administering an agent that causes growth cone collapse, the method of screening also includes using primary neurons from mammals, such as mice, that have been genetically manipulated to be null for a gene or genes that are involved in regulating growth cone collapse or neuron degeneration. For example, the method of screening includes the option of using primary neurons from mouse embryos that are null for the NPC1 gene. Because NPC1 is a regulator of cholesterol transport in a neuron, and because disruptions of cholesterol transport can lead to the activation of p38 MAPK, neurons prepared from npc−/− mice exhibit growth cone collapse. ul determining whether the test compound inhibits axon growth cone collapse based on whether pretreatment of neurons with the active agent solution causes a reduction in the number of neurons that undergo growth cone collapse.

### BRIEF DESCRIPTION OF THE TABLES

TABLE 1. Shows the effects of p38 MAPK and Mdm2 inhibitors on U18666A-induced changes in various proteins.

### BRIEF DESCRIPTION OF THE FIGURES

FIG. 1. Deregulation of p53 is associated with abnormal axonal development in neurons with genetically- or pharmacologically induced cholesterol transport perturbation. A and B. Immunofluorescence of p53 phosphorylated at Ser15 (p-p53, green) and E6-AP (red) in hippocampal neurons cultured from E18 npc1+/+ (A) or npc1−/− embryos (B) and kept for four days in vitro (DIV4). Scale bar=50 μm. C. Levels of p-p53 in axons and growth cones are decreased in DIV4 hippocampal neurons from npc1−/− mice. The X-axis shows the levels of p-p53 levels detected in growth cones (in black) and axons (in grey) as a percentage of the expression in the growth cones and axons of npc1+/+ mice. P-p53-immunoreactivity was quantified as described in Example 6 (n=30 growth cones; **p, 0.01 as compared to npc1+/+ mice). D and E. Over-expression of wild-type p53 blocks growth cone collapse induced by cholesterol transport inhibition. D. Hippocampal neurons prepared from wild-type mice were transfected at DIV3 with either a EGFP vector, a EGFP-wild-type p53 (EGFP-p53-wt) vector, or a EGFP-p53 with R175H mutation (EGFP-p53-mu) vector, and treated with 1 μM U18666A for 18 h. Neurons were then fixed and processed for immunostaining with anti-E6AP (red). Scale bar=20 μm. E. Quantitative analysis of U18666A-induced growth cone collapse in EGFP-p53-wt-transfected hippocampal neurons as compared to EGFP-vector transfected-hippocampal neurons. The X-axis shows the percentage of cells that exhibited growth cone collapse. n=30 growth cones from 3 individual experiments).

FIG. 2. Decreased axonal p-p53 immunoreactivity in the striatum of Npc1−/− mice. Immunofluorescent staining with anti-p-p53 (green) and anti-axon specific neurofilament (SIM-312; red) was performed on coronal brain sections from 2 week-old Npc1+/+ and Npc1−/− mice. In the striatum, p-p53 immunoreactivity was clearly reduced in axonal bundles containing axonal neurofilaments in Npc1−/− mice as compared to wild-types. p-p53 immunoreactivity was also present in oligodendrocytes. Scale bar=50 μm.

FIG. 3. Over-expression of wild-type p53 blocks U18666A-induced growth cone collapse. DIV3 hippocampal neurons from wild-type mice were first transfected with EGFP-vector (A), EGFPwild-type-p53 (p53-wt; B), or EGFP-mutant-p53 (p53-mu; C); 18 h later they were treated with 5 μM U18666A for two min before being processed for immunostaining with anti-E6AP antibodies (red). Scale bar=20 μm.

FIG. 4. U18666A treatment decreases cholesterol levels in axons and growth cones. Cultured hippocampal neurons were treated with 1 μM U18666A (D-F) or DMSO (A-C) for 18 h before being processed for immunostaining with anti-E6-AP (red in A&D) and -p-p53 (green in A and D, to label axons and growth cones) antibodies followed by filipin staining (blue in A and D). Panels B and E show filipin staining in axons while C and F show staining in cell bodies. Scale bar=20 μm.

FIG. 5. P38 MAPK activation is involved in growth cone collapse elicited by perturbation of cholesterol transport. A. Immunoblotting analysis of various proteins in cultured cortical neurons. Cortical neurons prepared from wild-type mice were treated at DIV4 with DMSO (D, vehicle), 5 μM U18666A (U), 5 μM U18666A plus p38 MAPK inhibitor, 1 μM SB203580 (U+S), or SB203580 alone (S). Shown are representative images of immunoblots probed with anti-phospho-Mdm2 (p-Mdm2), anti-phospho-p38 MAPK (p-p38), anti-phospho-p53 (p-p53, arrow), anti-RhoA, anti-phospho LIM Kinase (p-LIMK), anti-GAPDH (loading control), and anti-ubiquitin (Ubi) antibodies. U18666A treatment induced the appearance of a p-p53 immunopositive band (p-p53Δ) with a slightly smaller apparent molecular weight than native p-p53. B. Truncated p-p53 is not associated with axonal protein tau. Immunoprecipitation with Tau1 antibody or control IgG was performed as described in Material and Methods. Immunoprecipitated products and whole lysates (WL) were subjected to immunoblotting and blots were then probed with antibodies against total p53, p-p53 or tau. U18666A U18) treatment resulted in a marked increase in p-p53Δ in whole lysates compared to DMSO treated or non-treated (NT). C. Inhibition of p38 MAPK reduced cholesterol perturbation-induced growth cone collapse. Quantification of growth cone collapse in DIV4 hippocampal neurons treated with DMSO or U18666A in the presence or absence of SB203580 pre-treatment was performed as described at paragraph [0057] (**p, 0.01 as compared to DMSO-treated, ##p, 0.01 as compared to U18666A-treated; n=100 growth cones from three individual experiments). The X-axis shows the percent of hippocampal neurons exhibiting growth cone collapse. D. Quantitative analysis of p-p53 levels in axons and growth cones of DIV4 hippocampal neurons. (**p, 0.01 as compared to DMSO-treated and ##p, 0.01 as compared to U18666A-treated; n=25-40 growth cones from 3 individual experiments). The X-axis shows the levels of p-p53 detected in growth cones (black) and axons (grey)

FIG. 6. Localization of p38 MAPK and Mdm2 in axons and growth cones. DIV4 hippocampal neurons from wild-type mice were treated with DMSO or 5 μM U18666A for two min before being processed for immunofluorescence analysis of phosphorylated p38 (p-p38, green) and Mdm2 (p-Mdm2, green) distribution in axons and growth cones. Neurons were doubled immunostained with anti-E6AP antibodies (red). Inserts show enlarged images of growth cones. Scale bar=50 μm.

FIG. 7. Inhibition of p38 MAPK blocked U18666A-induced increase in RhoA expression in axons and growth cones. Wild-type hippocampal neurons were treated at DIV 4 with DMSO or U18666A in the presence or absence of SB203580 (SB) pre-treatment and processed for immunostaining with anti-RhoA (green) and anti-E6-AP (red) antibodies as described in Materials and Methods. A. Representative images. B. Quantitative analysis of RhoA levels in axons and growth cones (**p, 0.01 as compared to DMSO-treated and ##p, 0.01 as compared to U18666Atreated; n=25-40 growth cones from 3 individual experiments). The X-axis shows levels of RhoA detected in growth cones (black) and axons (grey).

FIG. 8. P38 MAPK specific siRNAs reduce U18666A-induced growth cone collapse. A. Hippocampal neurons cultured from wild-type mice were transfected with a set of siRNAs specific for p38 MAPK or control siRNAs and with a GFP vector at DIV3 and treated with U18666A at DIV 4 before being fixed and processed for immunostaining with anti-phospho-p38 (p-p38, red) antibodies. Inserts show enlarged images of growth cones. Application of p38 MAPK siRNAs, but not control siRNA, markedly reduced p-p38 immunoreactivity and U18666A-induced growth cone collapse. Results are representative of 3-4 culture dishes from 2 independent experiments. Scale bar=50 μm. B. Immunoblotting analysis of p38 knock-down by siRNA. Cortical neurons transfected with p38 MAPK specific or control siRNAs (CS) at DIV3 were collected on DIV4 and processed for immunoblotting with anti-total p38, -p-p38, or GAPDH (loading control). Treatment with p38 MAPK specific siRNAs, but not control siRNA reduced both total p38 and p-p38 by 90% as compared to non-treated (NT).

FIG. 9. Mdm2 activation is involved in U18666A-induced p53 degradation and growth cone collapse. A. Mdm2 inhibition blocked U18666A treatment-induced p-p53 truncation and ROCK activation. Cultured cortical neurons were treated with DMSO (D) or U18666A (U) in the presence or absence of pre-treatment with an Mdm2 inhibitor (M). Shown are representative images of immunoblots probed with anti-phospho-Mdm2 (p-Mdm2), anti-phospho-p53 (p-p53), anti-phospho-p38 MAPK (p-p38), anti-RhoA, anti-phospho LIM Kinase (p-LIMK), and anti-GAPDH (loading control) antibodies. Mdm2 inhibitor (Mdm2-In) blocked U18666A-induced increases in p-p53Δ, RhoA, and p-LIMK, but not in pMdm2 or p-p38. B and C. Mdm2 inhibition blocked U18666A treatment-induced growth cone collapse. DIV4 hippocampal neurons treated with DMSO or U18666A (U18) in the presence or absence of Mdm2 inhibitor pre-treatment (Mdm2-In) were processed for immunostaining with anti-p-p53 (green) and -E6AP (red) antibodies. B. Representative images. Scale bar=20 μm. C. Quantitative analysis of growth cone collapse. (**p, 0.01 as compared to DMSO treated, ##p, 0.01 as compared to U18666A treated; n=100 growth cones from 3 individual experiments). D. Quantitative analysis of p-p53 levels in axons and growth cones of DIV4 hippocampal neurons (**p, 0.01 as compared to DMSO-treated and ##p, 0.01 as compared to U18666A-treated; n=25-40 growth cones from 3 individual experiments).

FIG. 10. ROCK inhibition blocks U18666A-induced p-p53 decrease and rescues growth cones in hippocampal neurons cultured from wild-type mice. A. Immunofluorescence analysis of p-p53 (green) and E6-AP (red) distribution and growth cone morphology in cultured wildtype hippocampal neurons treated with DMSO or U18666A (U18) in the absence or presence of 10 μM Y-27632 (Y27). Scale bar=20 mm. B. Quantitative analysis of p-p53 levels in axons and growth cones of DIV4 hippocampal neurons (**p, 0.01 as compared to DMSO-treated and ##p, 0.01 as compared to U18666A-treated; n=25-40 growth cones from three independent experiments). C and D. Immunoblotting analysis of various proteins in cultured cortical neurons treated with DMSO (D) or U18666A (U) in the presence or absence of Y27632 (Y). C. Representative images of immunoblots probed with antibodies against ubiquitin (Ubi), phospho-Mdm2 (p-Mdm2), phospho-p38 MAPK (p-p38), phospho-p53 (p-p53), RhoA, phospho LIM Kinase (p-LIMK), and GAPDH (loading control). The X-axis shows the levels of p-p53 detected in growth cones (black) and axons (grey). D. Quantitative analysis of p-p53Δ, RhoA, and p-LIMK (**p, 0.01 as compared. to DMSO-treated; ##p, 0.01 as compared to U18666A-treated; n=3-6 from three individual experiments). The X-axis shows the levels of p-p53Δ (dark grey), RhoA (light grey), and p-LIMK (medium grey) detected in cortical neurons as a percentage of the respective proteins detected in DMSO-treated cortical neurons.

FIG. 11. ROCK inhibition with H1152 blocks U18666A-induced p-p53 decrease and rescues growth cones in cultured hippocampal neurons. Hippocampal neurons were treated at DIV4 with the ROCK inhibitor, H1152 (100 nM) for 3 h before being exposed to U18666A (U18, 5 μM) or DMSO for two min. Neurons were then subjected to immunofluorescence analysis of p-p53 (green) and E6-AP (red) distribution in axons and growth cones. Scale bar=20 μm.

FIG. 12. ROCK inhibition blocks U18666A treatment-induced decreases in “conformational mutant” p53 in axons and growth cones. DIV4 hippocampal neurons from wild-type mice were treated with DMSO or 5 μM U18666A for 2 min with or without pre-incubation with 10 μM Y27632. Neurons were then immunostained with anti-p-p53 (green) antibodies and a “conformational mutant” p53 specific antibody (mu-p53, red). Scale bar=20 μm.

FIG. 13. ROCK inhibition increases p-p53 levels and rescues growth cones in cultured hippocampal neurons from npc1−/− mice. A-C. Immunofluorescence of p-p53 (green) and E6-AP (red) in cultured npc12/2 hippocampal neurons treated with DMSO (A) or Y27632 (B). Scale bar=50 μm. High power images of growth cones are shown in C. Hippocampal neurons were prepared from E18 npc1−/− embryos and treated with 0.01% DMSO or 10 μM Y27632 (ROCK inhibitor) at DIV3 for 24 h before being processed for immunofluorescence staining. D. Quantitative analysis of p-p53 levels in axons and growth cones (n=30 growth cones; ##p, 0.01 as compared to values in DMSO-treated neurons from npc1−/− mice). The X-axis shows the levels of p-p53 detected in growth cones (black) and axons (grey) as percentage of p-p53 levels in DMSO-treated neurons.

FIG. 14. ROCK inhibition increases p-p53 and neurofilament immunoreactivity in striatal axons in developing npc1−/− mice. Immunostaining was performed with anti-p-p53 (green) and anti-neurofilament (SMI-312; red) antibodies in coronal brain sections from npc1+/+ or npc1−/− mice treated with vehicle or hydroxyfasudil monohydrochloride (npc1−/− HFD). A. Representative images containing fasciculated bundles in the caudoputamen. B. Quantification of levels of p-p53 and SMI-312 immunoreactivity in the coronal brain sections of (A). The X-axis shows the detected levels of p-p53 (black) and SMI-312 (grey). C. SMI-312 immunoreactive (SMI-312-ir) areas in the coronal brain sections of (A). ** indicates p, 0.01 compared to npc1+/+ mice and # and ## indicate p, 0.05 and 0.01 respectively compared to vehicle treated npc1 mice. Scale bar=50 μm.

FIG. 15. Inhibition of protein synthesis blocks U18666A-induced increase in RhoA and growth cone collapse. A and B. Immunoblotting analysis of various proteins in cultured cortical neurons treated with DMSO (D) or U18666A (U) in the presence or absence of the protein synthesis inhibitor ementine (Em). A. Representative images of immunoblots probed with antibodies against ubiquitin (Ubi), phospho-Mdm2 (p-Mdm2), phospho-p38 MAPK (p-p38), phospho-p53 (p-p53), RhoA, phospho LIM Kinase (p-LIMK), and GAPDH (loading control). B. Quantitative data of p-p53Δ, RhoA, and p-LIMK (**p, 0.01 as compared to DMSO-treated, ##p, 0.01 as compared to U18666A-treated; n=3-6 from three individual experiments). The X-axis shows the levels of p-p53Δ (dark grey), RhoA (light grey), and p-LIMK (medium grey) detected in cortical neurons as a percentage of the respective proteins detected in DMSO-treated cortical neurons. C. Emetine application also significantly reduced U18666A treatment-induced growth cone collapse (n=100 growth cones; **p, 0.001 as compared to DMSO-treated growth cones and ##p, 0.01 as compared to U18666A-treated). The X-axis shows the percent of cortical neurons exhibiting growth cone collapse. D. Treatment with p53 inhibitor, pifithrin-μ (P) induced rapid increase in levels of RhoA and p-LIMK; both events were blocked by emetine (E) treatment.

FIG. 16. Localization of phospho-4EBP1 in axons and growth cones. DIV4 hippocampal neurons from wild-type mice were treated with DMSO or 5 μM U18666A for two min before being processed for immunofluorescence analysis of phosphorylated 4EBP1 (p-4EBP1, green) and E6AP (red) distribution in axons and growth cones. Inserts show enlarged images of growth cones. Scale bar=50 μm.

FIG. 17. Potential signaling pathways involved in axonal pathology induced by genetic or pharmacological disruption of cholesterol homeostasis. A. p53 directly interacts with ROCK. Cortical neurons cultured from wild-type mice were collected at DIV4 and processed for immunoprecipitation (IP) with anti-mu-p53 antibodies (monoclonal made in mice) or control mouse IgG; immunoblots (IB) were probed with anti-p53 or anti-ROCK2 antibodies (both are rabbit polyclonal). WL, whole lysates. B. Perturbation of cholesterol transport, either genetically or pharmacologically, induces abnormal p38 MAPK activation, which then activates Mdm2 resulting in p53 degradation. p53 degradation disinhibits ROCK and stimulates local synthesis of RhoA leading to further increase in ROCK activation. ROCK phosphorylates and activates LIMK, leading to phosphorylation and inactivation of cofilin, which favors stabilization of filamentous actin (F-actin). On the other hand, numerous studies have shown that ROCK activation increases myosin light chain (MLC) phosphorylation through direct phosphorylation or indirectly through inhibition of MLC phosphatase-mediated dephosphorylation of MLC. Phosphorylation of MLC promotes its binding to F-actin and stimulates F-actin contraction, leading to growth cone collapse. Arrows indicate stimulation, while filled circles represent inhibition.

### DETAILED DESCRIPTION OF THE INVENTION

The invention relates to new therapeutic uses of Rho-kinase (ROCK) inhibitors. The invention, more particularly, relates to a method of treating neurodegenerative diseases (the method). This invention is not limited to the particular methodology, protocols, and reagents, etc., described herein and as such may vary. The terminology used herein is for the purpose of describing particular embodiments only, and is not intended to limit the scope of the present invention, which is defined solely by the claims.

Herein and in the claims, the singular forms “a,” “an,” and “the” include the plural reference and equivalents known to those skilled in the art unless the context clearly indicates otherwise. Other than in the operating examples, or where otherwise indicated, all numbers expressing quantities of ingredients or reaction conditions used should be understood as modified in all instances by the term “about.”

All patents and other publications that this disclosure identifies are incorporated herein by reference for the purpose of describing and disclosing. For example, the methodologies that such publications describe may be used in connection with the present invention, but are not to provide definitions of terms inconsistent with those presented herein. All statements as to the date or representation as to the contents of these documents are based on information available to the applicants, and do not constitute any admission as to the correctness of the dates or contents of these documents. Nothing in this regard should be construed as an admission that the inventors are not entitled to antedate such disclosure by virtue of prior invention or for any other reason.

All technical and scientific terms used herein have the same meaning as those commonly understood to one of ordinary skill in the art to which this invention pertains, unless the applicants provide an alternative definition. Although methods and materials similar or equivalent to those this disclosure describes herein can be used in the practice or testing of the present disclosure, suitable methods and materials are described below. The materials, methods, and examples are illustrative only and not intended to be limiting.

As stated above, the method of the invention relates to the use of a ROCK inhibitor in the treatment of neurodegenerative disorders. In various embodiments, the method treats neurodegenerative disorders that are associated with axon degeneration. However, the method is not limited to treating any particular disorder. For example, a non-limiting list of neurodegenerative disorders includes Alzheimer's disease, amyotrophic lateral sclerosis, Parkinson's disease, and Niemann-Pick type C disease. In certain embodiments, the method may be used to treat neurodegenerative disorders that are associated with the disruption of cholesterol metabolism. In other embodiments, the method may be used to treat neurodegenerative disorders that fall into the classification of lysosomal storage diseases, such as, but not limited to, Niemann-Pick type C (NPC) disease.

Regardless of the neurodegenerative disorder that is treated by the method, the method uses a pharmaceutical formulation that comprises a ROCK inhibitor as an active agent that can inhibit at least one intracellular signaling mechanism that mediates axon degeneration. In an embodiment, the method administers the formulation to a mammal such that the active agent contacts at least one central nervous system component of the mammal. Central nervous system components include, but are not limited to a sensory nerve, a motor nerve, an autonomic nervous system component, or an enteric nervous system component. The pharmaceutical formulation may be a solid or liquid dosage form as is known in the art which generally contains a therapeutically effective amount of the active agent, a pharmaceutically acceptable carrier and, optionally, one or more pharmaceutically acceptable excipients. Administration of the pharmaceutical formulation to a patient may be by any suitable means, for example, orally, such as in the form of a liquid, tablets, capsules, granules or powders; sublingually; bucally; parenterally, such as by subcutaneous, intravenous, intramuscular, or infusion techniques (e.g., as sterile injectable aqueous or non-aqueous solutions or suspensions); nasally, including administration to the nasal membranes, such as by inhalation spray; in dosage unit formulations containing non-toxic, pharmaceutically acceptable vehicles or diluents.

As discussed above, the method of the invention uses a Rho kinase (ROCK) inhibitor as the active agent. ROCK inhibitors are a known class of compounds. A ROCK inhibitor inhibits the functions of at least one isoform of ROCK, including, for example, ROCK I or ROCK II, and may inhibit more than one.

ROCK functions in various cellular activities, any of which may be inhibited by the ROCK inhibitor of the invention. For example, because one of ROCK's functions is to regulate actin organization, the inhibitor of the method can inhibit ROCK's ability to phosphorylate substrates substrates that are involved in actin organization. Such substrates may include, but are not limited to LIM kinase, myosin light chain (MLC), and MLC phosphatase. In various embodiments of the method, the ROCK inhibitor inhibits ROCK that has been activated by a member of the Rho kinase family. In certain embodiments of the method, the ROCK inhibitor inhibits ROCK that has been activated by Rho kinase that was expressed due to the degradation of p53. Thus, the method of the invention may inhibit ROCK activity that is the result of any upstream cell signaling event that causes p53 degradation. In certain embodiments, the method inhibits ROCK that became activated following the degradation of at least one p53 protein that had been directly associated with ROCK, or, alternatively, a member of a complex of proteins that included at least one ROCK protein.

Signaling events that lead to p53 degradation include, but are not limited to p38 MAPK activation of Mdm2, wherein activated Mdm2 triggers p53 ubiquitination and proteosomal degradation. Therefore, in certain embodiments of the invention, the ROCK inhibitor inhibits ROCK that was activated as a consequence of p38 MAPK activation. For example, p38 MAPK can result as a consequence of abnormal regulation of intracellular cholesterol transport. Accordingly, in an embodiment of the invention, the ROCK inhibitor inhibits ROCK that was activated as a consequence of abnormal cholesterol transport in at least one neuron. In certain of those embodiments, abnormal cholesterol transport can be caused by mutations in either, or both of the NPC1 or NPC2 genes.

With respect to the ROCK inhibitor of the method, it can be any pharmaceutically acceptable agent, or combination of agents, that is capable of inhibiting at least one isoform of ROCK, more in particular for inhibiting ROCK I and/or ROCK II isoforms. ROCK inhibition may be effected in vitro, in vivo, or both, and when effected in vivo, is preferably effected in a selective manner, as defined above. In various embodiments of the method, the ROCK inhibitor is trans-4-[(1R)-1-Aminoethyl]-N-4-pyridinylcyclohexanecarboxamide dihydrochloride, which is known commercially as Y-27632. In other embodiments of the method, the ROCK inhibitor is (S)-(+)-2-Methyl-1-[(4-methyl-5-isoquinolinyl)sulfonyl]-hexahydro-1H-1,4-diazepine dihydrochloride, which is known commercially as H 1152, or Fasudil Hydrochloride, which is alternatively known as HA 1077.

In another aspect, the invention features a method of screening for compounds that can be used for the treatment or prevention of neurodegenerative disorders, in particular disorders that are associated with axon degeneration. In general, the method of screening selects compounds that have an ability to inhibit at least one cell signaling pathway that regulates axon degradation. This screening method comprises (a) obtaining primary cultured mammalian neurons, (b) dividing the neurons into at least four cultures, (c) pretreating at least two of the neuron cultures by contacting the cultures with a solution of the test compound, (d) pretreating at least two of the neuron cultures by contacting the cultures with a first control solution, (e) treating at least one of the neuron cultures that has been pretreated with the test compound, and at least one of the neuron cultures that has been treated with the first control solution by contacting the neuron cultures with an agent that can trigger axon growth cone collapse (for example, U18666), and (f) determining whether the test compound inhibits axon growth cone collapse based on whether pretreatment of neurons with the active agent solution causes a reduction in the number of neurons that undergo growth cone collapse.

With respect to the primary cultured neurons of the aforementioned method of screening, the neurons can be prepared by using cell culture techniques that are well-known in the art. While primary neurons can be prepared from any tissues of a mammalian central nervous system, preferable tissues include neurons prepared from the cortex or the hippocampus regions of an embryonic mouse brain. In an embodiment of the screening method of the invention, cortical or hippocampal neurons are harvested from mouse embryos at embryonic day 18 (E18), and then cultured in NeuroBasal (GIBCO, Carlsbad, Calif.) with 10% bovine serum albumin (BSA), 2% B27, and 1% glutamine for three to four days before being used.

In some embodiments of the screening method of the invention, primary neurons can be prepared from central nervous system tissues from mice that are null for a particular gene or set of genes. Particularly useful gene deletions are those that encode protein that regulate axon degeneration or growth cone collapse, or both. For example, the deletion of the Niemann-Pick type C-1 gene in mice results in a phenotype that is characterized by neuronal growth cone collapse. Accordingly, primary neuron cultures that are prepared from mouse embryos which are null for the Niemann-Pick type C-1 gene (npc1−/− embryos) are useful in screening methods for identifying compounds that can prevent cultured neurons prepared from npc1−/− mice from undergoing growth cone collapse. Therefore, in various embodiments of the screening method of the invention, the method comprises (a) obtaining primary cultured neurons from npc1−/− embryos and npc1+/+ control embryos, (b) dividing the npc1−/− neurons into at least two separate cultures, and also dividing the npc1+/+ neurons into at least two separate cultures, (c) pretreating at least one of the npc1−/− neuron cultures, and at least one of the npc1+/+ neuron cultures with a solution of the test compound, (d) pretreating at least one of the npc1−/− neuron cultures, and at least one of the npc1+/+ neuron cultures with a solution of the test compound with a control solution, and (e) determining whether the test compound inhibits axon growth cone collapse in npc1−/− neuron cultures as compared to control solution-treated npc1−/− neuron cultures.

Further according to the aforementioned method of screening, potential test compounds can be any pharmaceutically acceptable compound that one of skill in the art suspects could be used to treat neurodegenerative disorders and diseases. In various embodiments of the method of screening, test compounds can be selected based on their known cell signaling targets. A non-limiting list of signaling targets may include, for example, p38 MAPK, Mdm2, small GTPase proteins such as RhoA, Rho associated kinase (ROCK1 and ROCK2), cholesterol transport proteins, MLC phosphatase, MLC, and LIMK. Typically, one of skill in the art also solubilizes test compounds, and decides on their initial dosage ranges according to protocols and knowledge in the art.

Pretreatment of neuronal cultures with the test compound generally involves a pretreatment period before the addition of an agent that induces axon degeneration or growth cone collapse. Similarly, neuron cultures that naturally undergo growth cone collapse or neuron degeneration because the neurons were prepared from mice that are null for a gene that regulates an aspect of axon structure, may also require a minimum length of treatment time to rescue neurons from growth cone collapse and axon degeneration. Generally, time periods of two hours or less are sufficient to allow potentially effective test compounds to react with their signaling pathway targets, and functionally inhibit growth cone collapse or axon degeneration. However, some test compounds may function in mere seconds or minutes after being added to a neuron culture, whereas other test compounds may require up to twenty four hours to functionally inhibit growth cone collapse or axon degeneration.

Growth cone collapse can be measured in the aforementioned method of screening by relying on confocal microscopy to visualize and quantify growth cone collapse. Typically, images are taken by preferably using a 60× oil-immersion objective. However, the magnification of the objective that is used to view growth cones is to be chosen at the discretion of the microscope operator. Normally, about 20-30 images are randomly selected from a single 20 mm culture dish, and at least four to six dishes from three to six independent culture preparations/experiments are used for each experimental group. Within an experiment, cultures used for different experimental groups and designed for comparison are stained simultaneously and imaged with the same acquisition parameters. Quantification is to be performed blindly by multiple researchers. Growth cones with less than 1 filopodium are considered collapsed.

### EXAMPLES

The following materials and methods were used.

Animals. A breeding colony of Npc1″ heterozygous mice that were on a BALB/c background (Jackson Laboratory, Bar Harbor, Me.) was established in order breed wild type (npc+/+) and npc1−/− mice. The genotype of the mice was determined by employing a polymerase chain reaction (PCR)-based method as described in reference [3]. The Institutional Animal Care and Use Committee (IACUC) of Western University of Health Sciences approved the care and experimental protocols that these examples describe. The National Institutes of Health Guide for the Care and Use of Laboratory Animals and Animal Husbandry governed the use of animals by the inventors.

Neuronal cultures. Cortical and hippocampal neurons were prepared from npc1+/+ and npc1−/− embryos at embryonic day 18 (E18); time-pregnant wild-type BALB/c or npc1+/− mice were obtained either from Charles River Laboratories (San Diego, Calif.) or from our breeding colony respectively. Neurons were cultured in NeuroBasal (GIBCO, Carlsbad, Calif.) with 10% bovine serum albumin (BSA), 2% B27, and 1% glutamine for three to four days before being used.

Chemicals and antibodies. (R)-(+)-trans-N-(4-Pyridyl)-4-(1-aminoethyl)-cyclohexanecarboxamide (Y27632) and H1152 (ROCK inhibitors), trans-4-Iodo, 49-boranyl-chalcone (Mdm2-inhibitor), SB203580 (p38 inhibitor), emetine (protein synthesis inhibitor), and Control rabbit serum, anti-E6-AP, anti-ubiquitin and anti-ROCK2 antibodies were from Sigma (St. Louis, Mich.). Anti-RhoA and anti-p53 antibodies were from Santa Cruz Biotechnology (Santa Cruz, Calif.). Anti-GAPDH antibody was from Millipore (Billerica, Mass.). Anti-phospho-Mdm2 (Ser166), anti-phospho-p53 (Ser15), antu-phosphor-4EBP1 (Thr37/46), anti-phospho-LIMK1, 2(Thr508/505), anti-phospho-p38 MAPK (Thr180/Tyr182), anti-p38 MAPK antibodies and a p38 MAPK siRNA kit (SignalSilenceH) were from Cell Signaling Technology (Danvers, Mass.). Mutant conformation specific p53 (Mu-p53) antibody, Alexa488 conjugated anti-rabbit and Alexa594 conjugated anti-mouse antibodies were from Invitrogen (Carlsbad, Calif.). Expression plasmids and transfection. The construction of the p53 and p53-R175H expression plasmids that were used in these examples were described in reference [17]. Plasmid transfection was performed as also previously described in reference [17]. Briefly, neurons were incubated with DMEM (HyClone, Logan, Utah) with the addition of (per ml) 1 μg plasmid DNA, 40 μl 0.25 M CaCl₂, and 41 μl BES (pH 7.1) for 3 h. Cultured medium was then replaced with fresh medium and neurons were further cultured for 18 to 24 h before being processed for time-lapse imaging experiments or immunostaining analysis.

Treatment. For primary cultured neurons, chemicals (U18666A and inhibitors of various enzymes) were first dissolved in 10% DMSO before being diluted in cultured medium; final DMSO concentration was lower than 0.01%. For in vivo treatment, hydroxyfasudil monohydrochloride (Sigma) was dissolved in double-distilled H₂O and injected subcutaneously at 10 mg/kg, twice a day from postnatal day 7 to day 28.

Immunofluorescent staining. Hippocampal neurons were fixed with 4% paraformaldehyde in phosphate buffer (PB; pH 7.4) for 15 min. After washing with 1× phosphate buffer saline (PBS), cells were permeabilized with 0.05% Triton X-100 in 1×PBS for 15 min, and incubated with blocking buffer (3% BSA, 0.02% Triton X-100 in 1×PBS) for 15 min before being probed with primary antibodies. The following primary antibodies were used: anti-E6AP (1:1000), anti-phospho-p53 (1:250), anti-phospho-4EBP1 (1:1000), antiphospho-Mdm2 antibody (1:250), anti-phospho-p38 antibody (1:250), anti-RhoA antibody (1:1500). All primary antibodies were diluted in blocking buffer and incubated at 4° C. for 18 h. After six washes (6×10 min) with 1×PBS at room temperature, cells were incubated with secondary antibodies, Alexa488-anti-rabbit (1:500) or Alexa594-anti-mouse (1:500); both antibodies were diluted in blocking buffer and incubated at room temperature for one h. Cells were then washed with 1×PBS (6×10 min) and sealed with mounting medium (Vectashield; Vector Laboratories, Inc., Burlingame, Calif.) containing 49,69-diamidino-2-phenylindole (DAPI) to stain nuclei. Immunofluorescent signal was detected with a Nikon confocal microscope (Nikon TE 2000U with DEclipse C1 system; Melville, N.Y.).

Filipin staining. Filipin has been demonstrated to specifically stain free cholesterol since treatment with cholesterol oxidase results in a complete loss of fluorescence [19]. After immunostaining with anti-E6-AP and anti-p-p53 antibodies and corresponding secondary antibodies conjugated with either Alexa Fluor H 594 or Alexa Fluor H 488, neurons were washed with 1×PBS and incubated in the dark with 375 mg/ml filipin in 1×PBS for 2 h at room temperature. Neuronal cultures were then washed again with 1×PBS before being examined with confocal microscopy.

Perfusion and Immunohistochemistry. Mice were perfused with freshly prepared 4% paraformaldehyde in 1×PBS. Brains were then removed and post-fixed in 4% paraformaldehyde for 16 h followed by incubation with graded sucrose solutions. Brains were sectioned into 30 μm coronal sections with a microtome. Floating sections were processed for immunostaining as described previously [7]. Briefly, sections were incubated with rabbit anti-p-p53 (1:250) and mouse anti-pan axonal neurofilament (SMI-312, 1:500; Covance) antibodies in 5% horse serum diluted in 0.1M PB overnight at 4° C. After three washes, sections were incubated with Alexa Fluor H 488 conjugated goat anti-rabbit and Alexa Fluor H 594 conjugated goat anti-mouse secondary antibodies. After four more washes, sections were then mounted onto SuperfrostH plus slides (VWR, West Chester, Pa.) and confocal images were acquired by using the Nikon microscope. Quantification of p-p53 and neurofilament immunoreactivity in fasciculated bundles in the striatum was performed by using NIH ImageJ software. Briefly, images of the caudoputamen from different animals were taken at the same coronal level using the same acquisition parameters. Analyzed area consisted of 450 μm×420 μm that was taken from two sections per mouse; three different mice were used for each experimental group. Means of integrated density and areas were quantified and expressed as percentage of values from npc1+/+ mice.

Quantification of growth cone morphology and immunoreactivity. Confocal images were taken using the 60× oil-immersion objective. About 20-30 images were randomly selected from each culture dish (20 mm in diameter); at least four to six dishes from three to six independent culture preparations/experiments were used for each experimental group. Within an experiment, cultures used for different experimental groups and designed for comparison were stained simultaneously and imaged with the same acquisition parameters. Quantification was done blindly by multiple researchers. Growth cones with less than 1 filopodium were considered collapsed; 100 growth cones were-quantified for each experimental group. Image J software was used to quantify immunoreactivity intensity of p-p53 and RhoA in axons and growth cones; the “total integrated density” was used instead of “average intensity”. Briefly, individual growth cones were outlined manually and the total integrated density was measured using Image J software. For quantification of immunoreactivity in axons, a 50 μm fragment of axons from the neck of growth cones towards the cell body was selected and integrated density measured.

Immunoprecipitation and immunoblotting procedures. For immunoprecipitation, cultured cortical neurons were lysed in lysis buffer [0.05 M Tris base, 0.9% NaCl, pH 7.6, and 0.5% Triton X-100 plus Protease Inhibitors Cocktail (1:100; EMD Biosciences) and phosphatase inhibitor cocktails (1:500; Sigma)]. Lysates were centrifuged at 16,000×g for 30 min at 4° C. Supernatant were then cleared with a mixture of protein A/Gagarose beads (each 50%) for 1 h at 4 uC, and after a brief spin, pellets were discarded. A small portion of the supernatants was used as input. The reminder of the supernatant was immunoprecipitated overnight with control IgG or Tau1 antibodies. Immunoprecipitates were captured by incubation with protein A/G-agarose beads for 3 h at 4° C. After several washes, the beads were resuspended in 2×SDS sample buffer [4% sodium dodecyl sulfate (SDS), 100 mM Tris-HCl (pH 6.8), 10% b-mercaptoethanol, 20% glycerol and 0.2% bromophenol blue] and boiled for 10 min. The resulting proteins were separated by SDS-PAGE, and transferred to polyvinylidene difluoride membranes for immunoblotting using previously described protocols [17].

Statistics. All experiments were performed at least 3 times with independent culture preparations. Results were expressed as means±SEM, and p values were determined by one-way ANOVA followed by post-hoc analysis; p values less than 0.05 were considered statistically significant.

### Example 1

### Increased Growth Cone Collapse and Decreased Levels of Phosphorylated p53 in Hippocampal Neurons Cultured from npc1−/− Mice

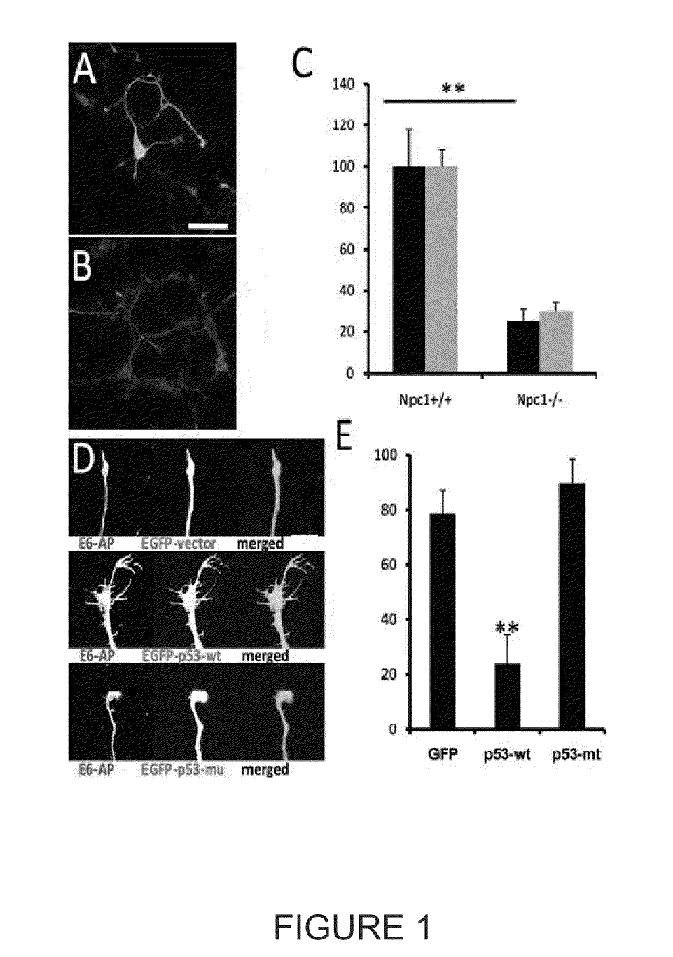

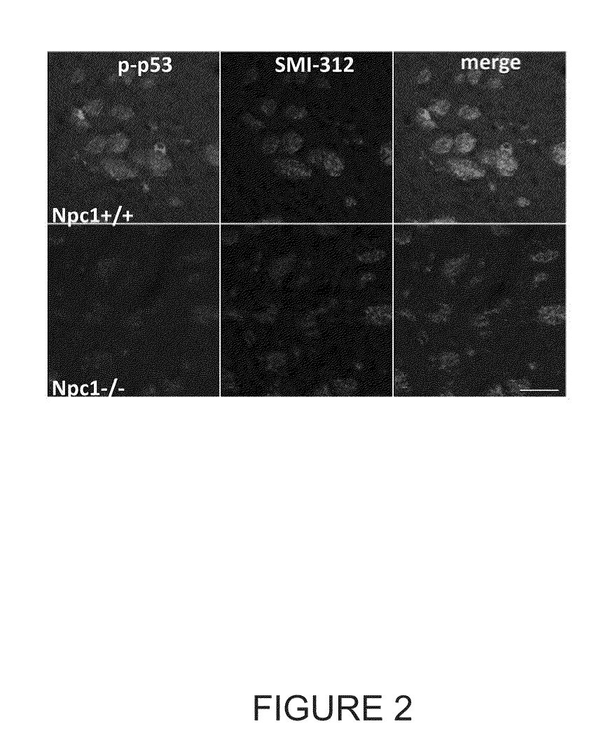

Hippocampal neurons from E18 npc1−/− and npc1+/+embryos were cultured for four days in vitro (DIV) and processed for double-immunofluorescent staining with antibodies against E6-AP (an E3 ligase), and phosphorylated p53 (p-p53). Both proteins were highly expressed in axons and growth cones, as previously reported [17]. In cultured neurons from npc1+/+mice, high levels of p-p53 were observed mainly in cell bodies, axons and growth cones (FIG. 1A, green), whereas in those from npc1−/− mice, only low levels of p-p53 were found and mainly in cell bodies (FIG. 1B). Cultured hippocampal neurons from npc1−/− mice exhibited a much higher rate of growth cone collapse (78±2% vs 8±2%; n=100 growth cones, p, 0.01) with small growth cones and few or no filopodia, as compared to those from npc1+/+ mice. Quantitative analysis indicated that levels of p-p53 immunoreactivity in axons and growth cones were decreased by about 80% as compared to wildtype values (FIG. 1C). Decreased axonal levels of p-p53 were also observed in brain tissues from 2-week old npc1−/− mice, especially in the striatum (FIG. 2).

### Example 2

### U18666A-Induced Growth Cone Collapse was Blocked by Over-Expression of Wild-Type p53

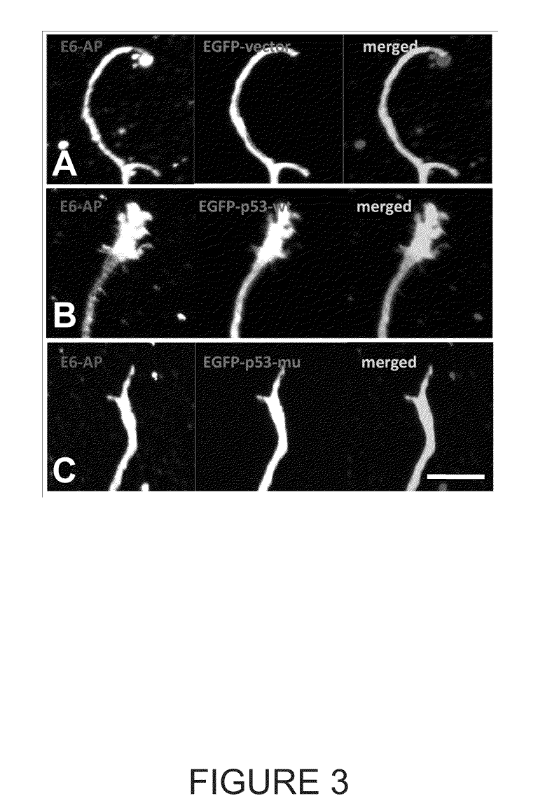

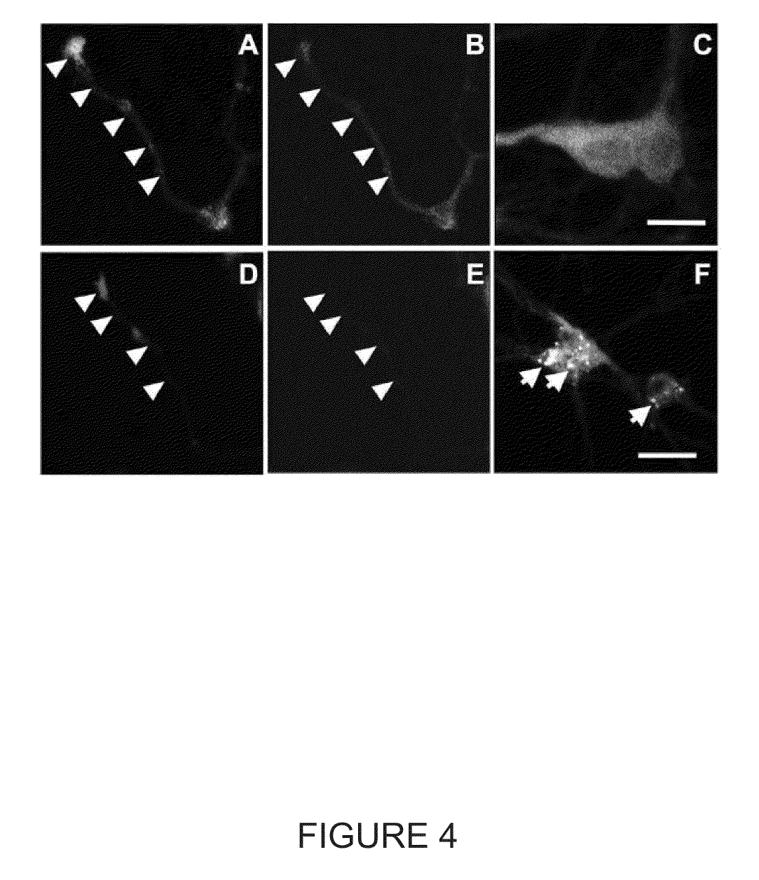

Because axonal growth cone collapse in hippocampal neurons from npc1−/− mice was associated with decreased levels of p-p53, over-expression of wild-type p53 was tested to determine if it could reverse growth cone collapse. In this set of experiments, the amphiphilic amine and cholesterol transport inhibitor, U18666A, was used to induce a NPC-like phenotype in hippocampal neurons cultured from wild-type mice. U18666A has been used to induce NPC-like phenotype in various cultured cells, including neurons [18]. Treatment with 1 μM U18666A for 18 h induced growth cone collapse in about 80% of hippocampal neurons prepared from wild-type mice and transfected with an EGFP-vector or a vector containing p53 with the R175H mutation (p53-R175H), a conformational mutation that is frequently found in tumor cells that lack p53 function (FIGS. 1D and E). The same treatment resulted in only 20% growth cone collapse for neurons transfected with wild-type p53 (FIGS. 1D and E). Wild-type p53 transfection also blocked growth cone collapse elicited by short-time (2 min) treatment with a higher concentration of U18666A (5 μM) (FIG. 3). It was previously shown that p53-R175H proteins form aggregates in cell bodies and failed to be targeted to axons and growth cones in cultured hippocampal neurons [17]. To verify that treatment with 1 μM U18666A for 18 h disrupted cholesterol distribution, hippocampal neurons were stained with filipin, a fluorescent probe that has been widely used to stain cholesterol [19]. In vehicle-treated controls, filipin fluorescence was observed in cell bodies, axons (arrowheads), and growth cones (FIG. 4, A-C). In U18666A-treated neurons, a marked decrease in fluorescence intensity was observed in axons and collapsed growth cones (FIGS. 4, D and E) in concurrence with the appearance of intensely labeled granules that resembled late endosomes/lysosomes in cell bodies (arrows in FIG. 4F).

### Example 3

### P38 MAPK and Mdm2 Activation Participated in U18666A Treatment-Induced p-p53 Degradation and Growth Cone

P53 levels are tightly regulated in cells by a negative feed-back loop between p53 and Mdm2, a p53 target gene. Mdm2 activation results in p53 degradation. The roles of p38 MAPK and Mdm2 in the regulation of p53 levels and growth cone morphology were analyzed.

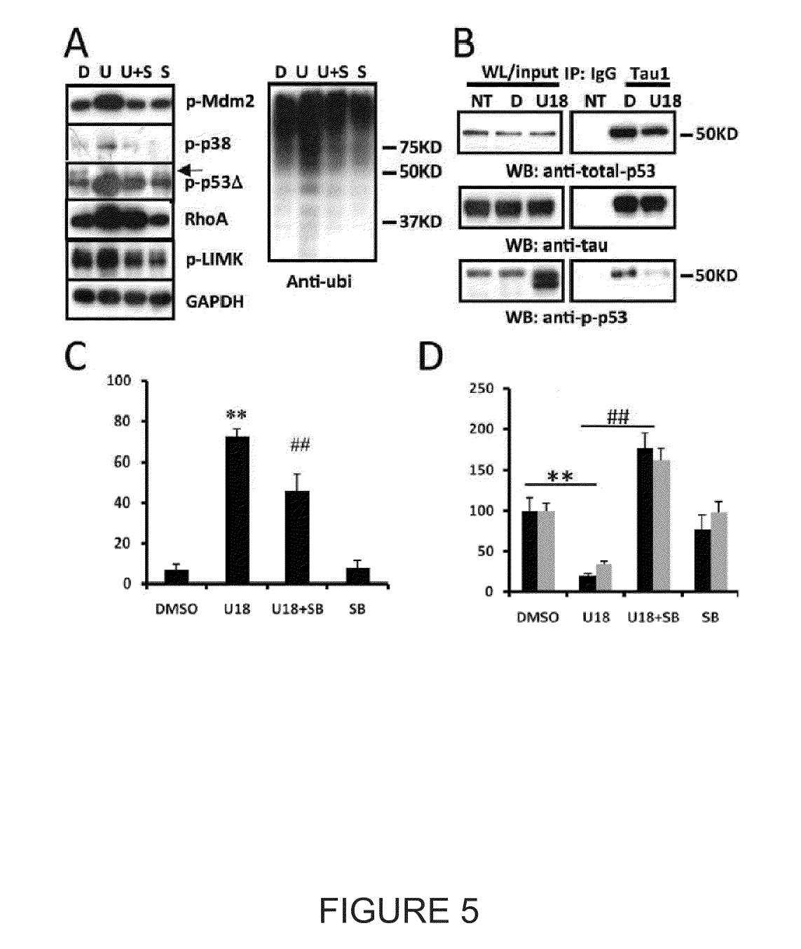

Immunoblot analysis indicated that treatment of wild-type cortical neurons at DIV4 with 5 μM U18666A for 2 min induced a rapid decrease in p-p53 levels (arrow in FIG. 5A) with a corresponding increase in levels of a p-p53 immunopositive band with a slightly smaller molecular weight (thereafter referred to as p-p53 breakdown product, p-p53Δ) than in control samples. Because the p-p53Δ and p-p53 bands were very close in immunoblots and the former was the predominant band, p-p53Δ levels were used as an index of p-p53 degradation. Immunoprecipitation was used to determine whether p-p53 truncation affected its association with the microtubule-associated protein tau, a protein that is abundantly and exclusively expressed in axons. p53 labeled by either anti-p-p53 or anti-p53 antibodies was immunoprecipitated by Tau1 antibodies (FIG. 5B). U18666A treatment of the wild-type cortical neurons resulted in a marked increase in p-p53Δ levels in whole lysates, but p-p53Δ was absent in Tau1 pull-down products.

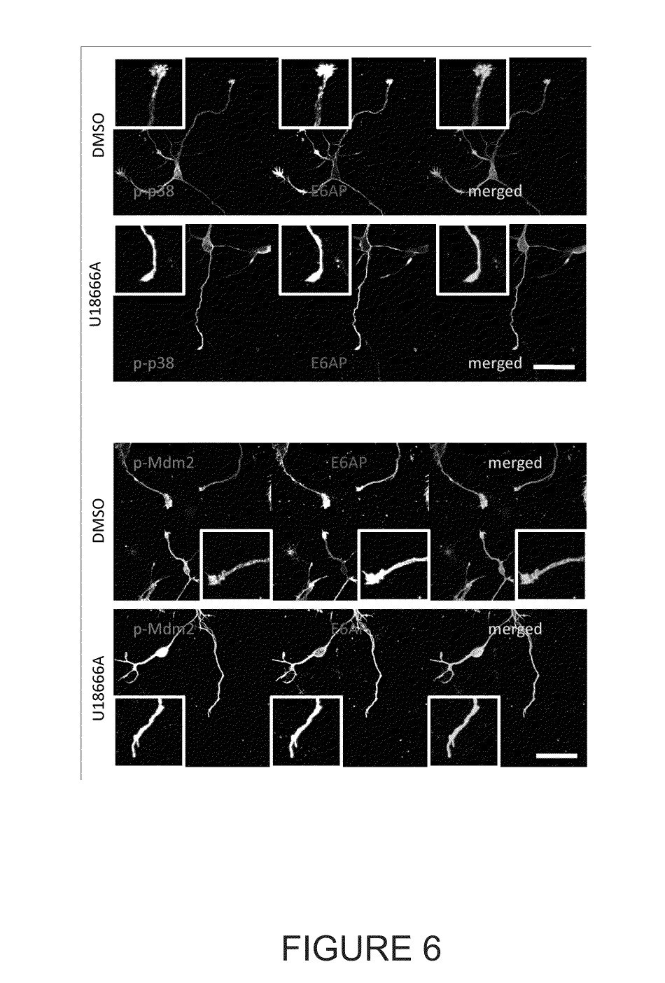

Immunoblot results also showed that U18666A treatment of wild-type cortical neurons markedly increased levels of Mdm2 phosphorylated at Ser166 (p-Mdm2 hereafter; FIG. 5A). Increased levels of p-p53Δ and p-Mdm2 were quantified using the ImageJ program (National Institutes of Health, Bethesda, Md.). The quantified levels are contained in Table 1 below. Levels of the dual-phosphorylated p38 MAPK (Thr180/Tyr182, hereafter referred to as p-p38), the active form of the enzyme [23,24], were also increased in U18666A-treated neurons as compared to vehicle-treated (FIG. 5A), while levels of the non-phosphorylated p38 MAPK were not altered (Table 1). Immunofluorescent staining performed with antibodies against p-p38 and p-Mdm2 indicated that levels of these phosphoproteins were increased in axons and growth cones in U18666A-treated neurons, as compared to vehicle-treated controls (FIG. 6). U18666A treatment also increased levels of RhoA and phosphorylated Lim kinase (p-LIMK) (FIG. 5A).

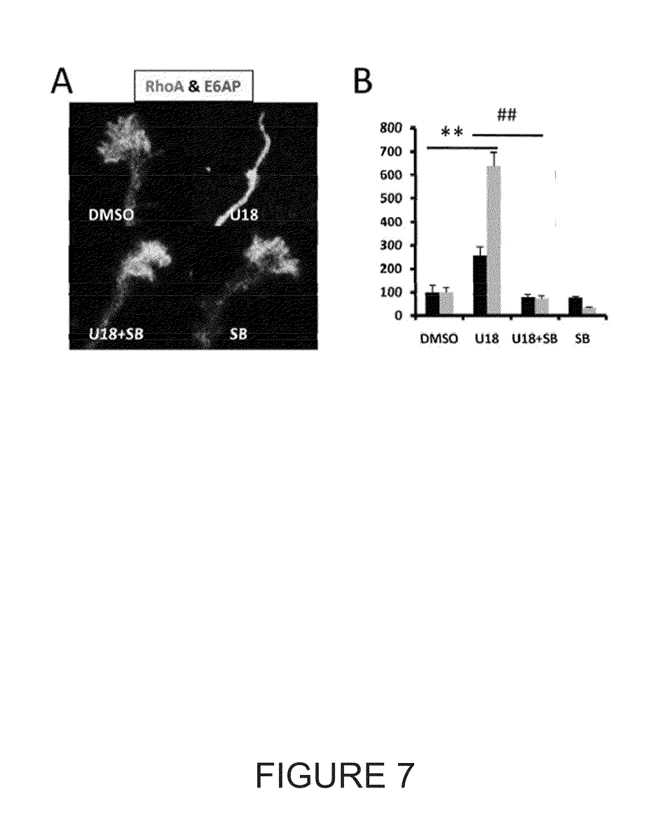

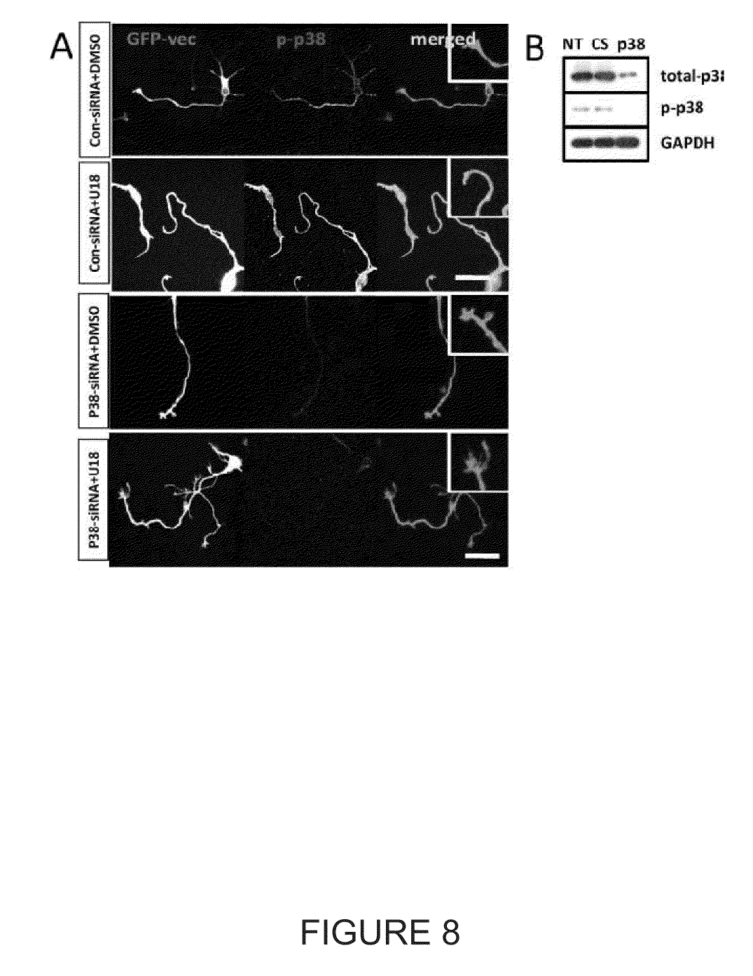

The p38 MAPK inhibitor, SB203580, was used in experiments designed to determine the extent to which p38 MAPK activation was involved in U18666A-induced growth cone collapse. Preincubation of cultured neurons with 1 μM SB203580 for 2 h before treatment with U18666A significantly reduced growth cone collapse elicited by U18666A (FIG. 5C). P38 MAPK inhibition also markedly reduced Mdm2 and p38 MAPK phosphorylation, p-p53 degradation, and RhoA increase resulting from U18666A treatment. The blocking effects of SB203580 on p-p53 truncation (FIG. 5D) and RhoA increase (FIGS. 7, A and B) in axons and growth cones were even more evident when analyzed with immunohistochemistry. Immunoblots probed with anti-ubiquitin (Ubi) and anti-p-LIMK antibodies indicated that p38 MAPK inhibition also reduced U18666A treatment-induced increases in protein ubiquitination and LIMK phosphorylation (FIG. 5A and Table 1). U18666A-induced growth cone collapse was also blocked by a set of siRNAs specific for p38 MAPK but not by control siRNAs, which further confirmed the involvement of this kinase in this process (FIG. 8). P38 MAPK siRNAs alone did not significantly modify growth cone morphology (FIG. 8A). The reduction of p-p38 levels by siRNA treatment was also confirmed by immunoblotting (FIG. 8B).

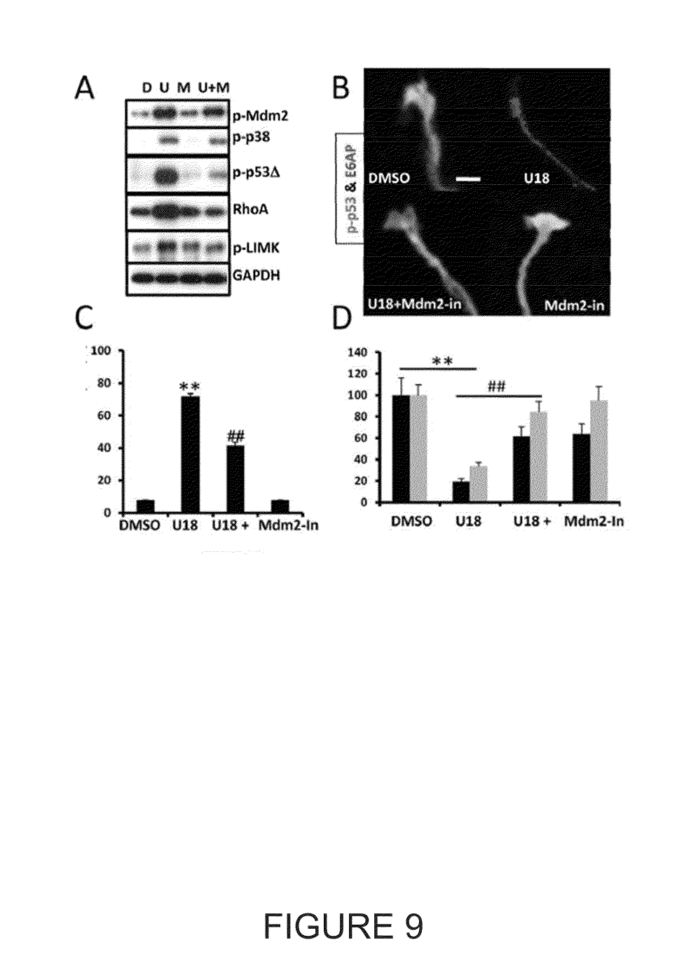

The critical role of Mdm2 in U18666A-induced growth cone collapse was further tested with an Mdm2 specific inhibitor (Mdm2-in); pretreatment with 1 μM Mdm2-in significantly reduced U18666A-induced growth cone collapse (FIGS. 9, B and C; p, 0.01, n=100 growth cones). Immunoblotting results showed that the Mdm2 inhibitor also significantly reduced the increase in p-p530 (FIG. 9A; Table 1). Image analysis indicated that the Mdm2 inhibitor significantly reduced U18666A-induced decrease in p-p53 levels in axons and growth cones (FIG. 9D; p, 0.01, n=25-40 neurons). Mdm2 inhibition also blocked U18666A-induced increase in RhoA levels (FIG. 9A). Immunohistochemical analysis showed that in U18666A plus Mdm2 inhibitor-treated neurons, RhoA levels in axons and growth cones were reduced from 640±56% to 67±16% and 257±37% to 138±24% (mean±SEM; p, 0.01 when compared to U18666A-treated; n=25-40 from three individual experiments, RhoA levels reported as expressed as a percentage of the RhoA levels in control, vehicle-treated neurons), respectively. Mdm2 inhibitor alone did not significantly change RhoA expression in either axons (102±15%) or growth cones (136±20%). Mdm2 inhibition did not alter U18666A-induced phosphorylation of either Mdm2 or p38 (FIG. 9A).

TABLE 1

| | DMSO | U18666A | | U18666A + SB203580 | | SB203580 | U18666A + Mdm2_In | | Mdm2-In |
|---|---|---|---|---|---|---|---|---|---|
| p-Mdm2 | 100 | 549 ± | 21** | 100 ± | 2^{##} | 92 ± 1 | 476 ± | 8 | 100 ± 4 |
| p-p38 | 100 | 493 ± | 24** | 99 ± | 3^{##} | 96 ± 24 | 472 ± | 19 | 108 ± 17 |
| t-p38 | 100 | 107 ± | 1 | 98 ± | 3 | 99 ± 5 | 107 ± | 1 | 104 ± 0 |
| p-p53Δ | 100 | 775 ± | 29** | 97 ± | 6^{##} | 94 ± 6 | 280 ± | 23^{##} | 106 ± 2 |
| t-p53 | 100 | 94 ± | 1 | 103 ± | 1 | 107 ± 2 | 98 ± | 4 | 104 ± 1 |
| Ubiquitin | 100 | 435 ± | 14** | 93 ± | 6^{##} | 95 ± 7 | 117 ± | 5^{##} | 102 ± 5 |
| RhoA | 100 | 418 ± | 16** | 216 ± | 21^{##} | 96 ± 0 | 114 ± | 6^{##} | 105 ± 1 |
| p-LIMK | 100 | 411 ± | 15** | 100 ± | 3^{##} | 100 ± 3 | 147 ± | 10^{##} | 117 ± 6 |
| p-4EBP1 | 100 | 389 ± | 7** | 276 ± | 19^{#} | 99 ± 3 | 327 ± | 3^{##} | 104 ± 2 |

**p < 0.01 as compared to DMSO-treated;
#p < 0.05 and ##p < 0.01 as compared to U18666A-treated;
n = 3-6 from 3 individual experiments.
dol: 10.1371/journal.pone.0009999.t001

### Example 4

### ROCK Inhibition Reduced U18666A-Induced Growth Cone Collapse and p-p53 Truncation

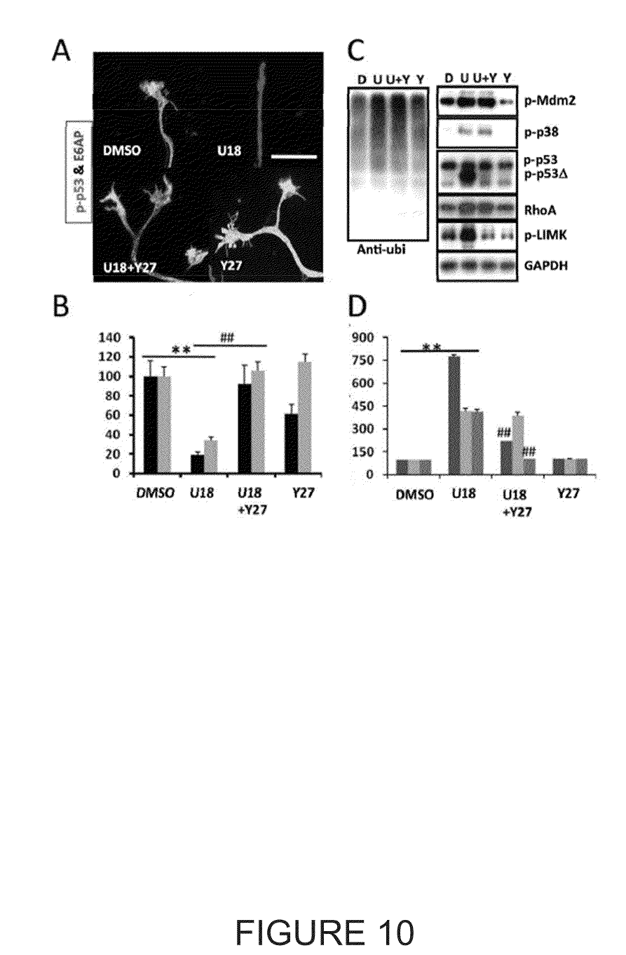

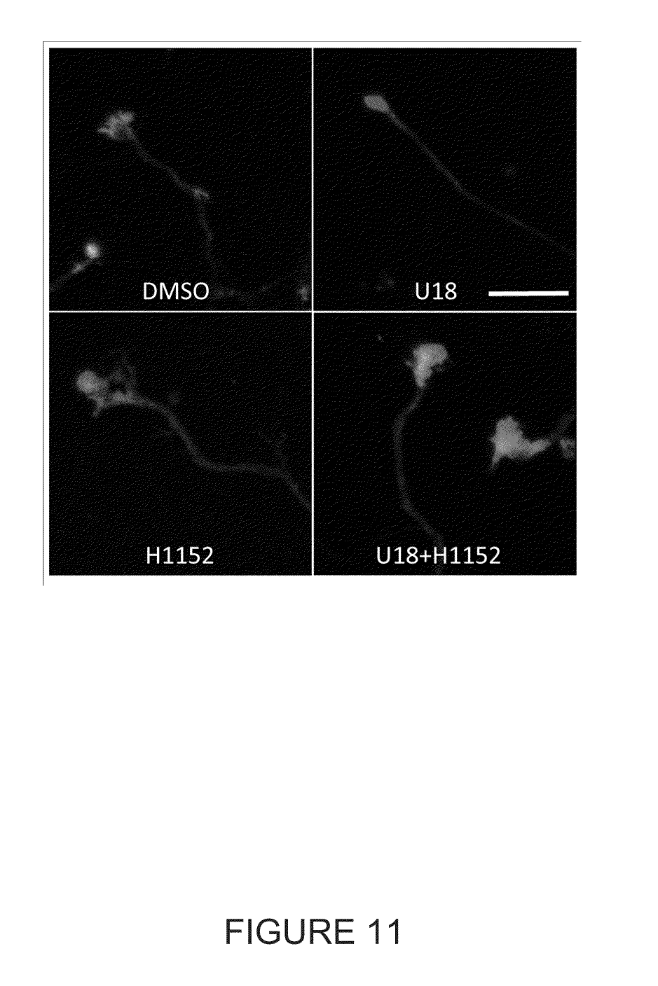

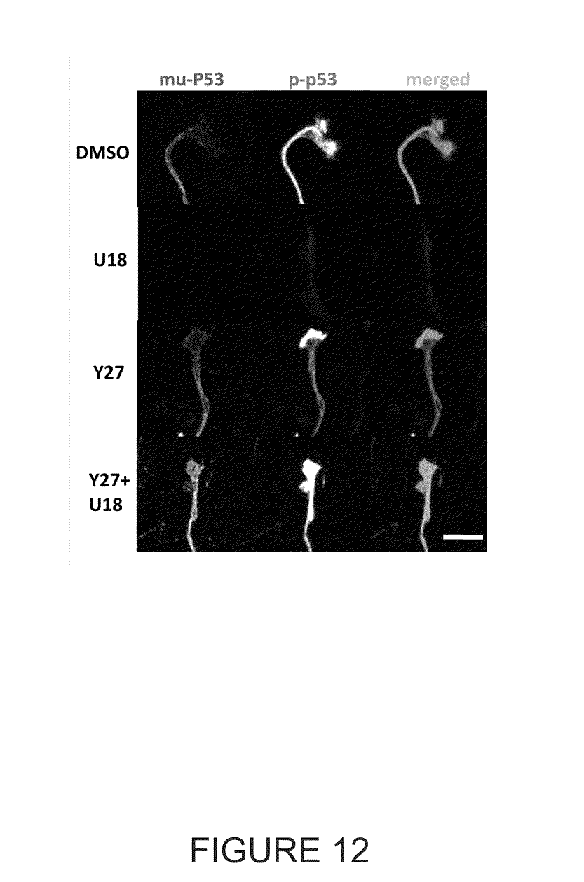

Rho kinase is involved in growth cone collapse. Moreover, growth cone collapse can be induced by inhibition of p53 with pifithrin-μ, can be rescued by ROCK inhibitors [17]. Immunoblotting and immunohistochemical analysis showed that U18666A treatment induced a marked increase in RhoA levels, which was blocked by inhibition of p38 MAPK and Mdm2. To further test the role of the Rho-ROCK signaling pathway in U18666A-induced growth cone collapse, cultured neurons were pre-treated with the widely used specific ROCK inhibitor, Y-27632. Pre-incubation of wild-type hippocampal neurons at DIV4 with Y-27632 (10 μM) for 2 h before treatment with 5 μM U18666A for 2 min significantly reduced U18666A-induced growth cone collapse (FIG. 10A; 41±1% vs. 72±2%; p, 0.01, n=100 growth cones). Y-27632 pretreatment also reversed U18666A-induced decrease in p-p53 immunoreactivity in axons and growth cones (FIGS. 10, A and B). Similar results were obtained following pre-treatment with 1 μM Y-27632. The involvement of ROCK was further tested by using another inhibitor, H 1152. Pre-treatment with 100 nM H 1152 for 3 h also blocked U18666A-induced growth cone collapse and decrease in p-p53 immunoreactivity (FIG. 11). Immunoblotting results indicated that pre-treatment with Y-27632 did not block U18666A-induced increase in p38 and Mdm2 phosphorylation, protein ubiquitination and RhoA levels (FIGS. 10, C and D), but significantly reduced U18666A-induced increase in levels of phosphorylated LIM kinase, an enzyme downstream of ROCK (p-LIMK; FIG. 10C). ROCK inhibition also significantly reduced p-p53 truncation. Treatment with U18666A markedly reduced levels of “mutant” p53 in axons and growth cones, an effect also blocked by Y-27632 (FIG. 12).

### Example 5

### ROCK Inhibition Reduced Axonal Abnormality of npc1−/− Mice In Vitro and In Vivo

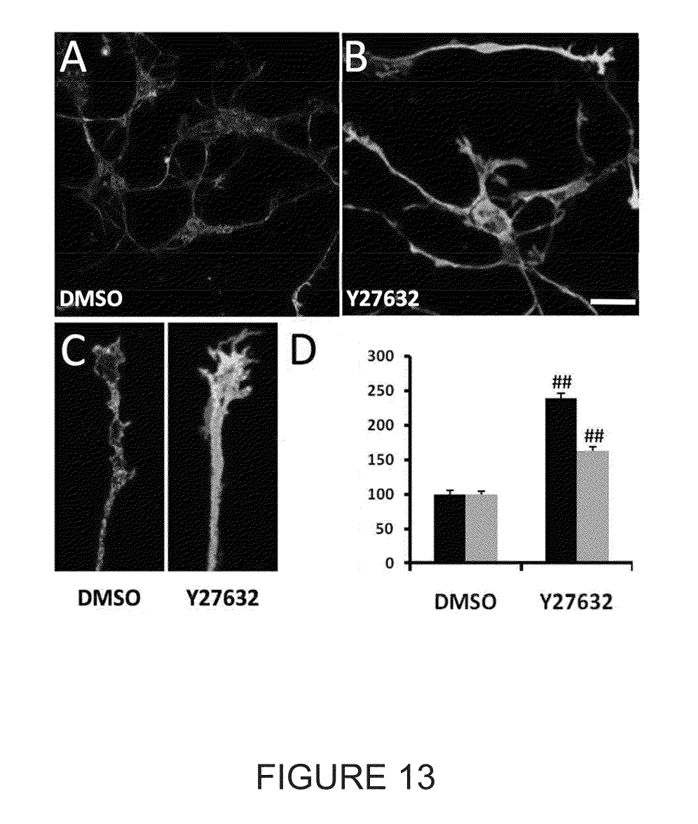

The question of whether ROCK activation is involved in spontaneous growth cone collapse in neurons with genetic Npc1 deficiency was addressed. Hippocampal neurons cultured from npc1−/− mice were treated for 18 h with vehicle or 10 μM Y-27632 at DIV3. ROCK inhibition significantly reduced growth cone collapse (48±1% vs. 80±2%; p, 0.01, n=100 growth cones) and increased p-p53 immunoreactivity in axons and growth cones in hippocampal neurons cultured from npc1−/− mice (FIG. 13).

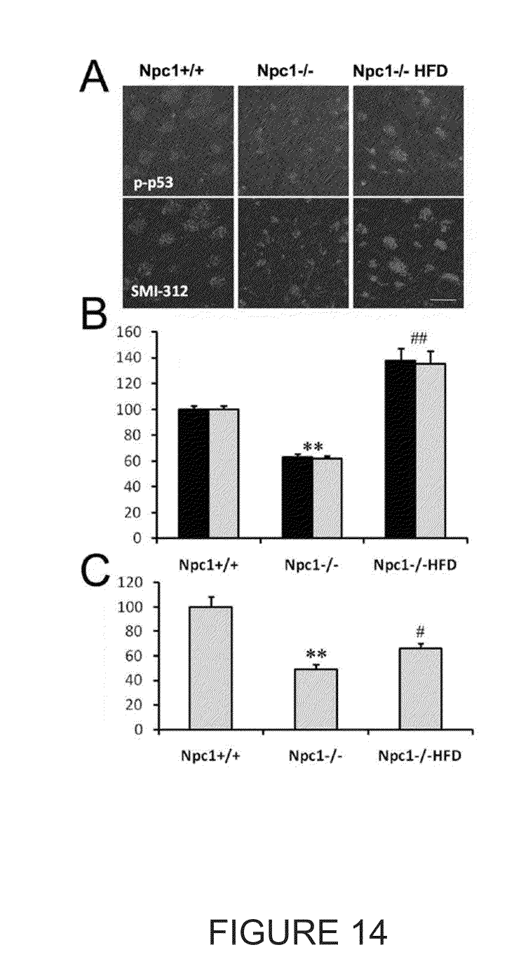

Another ROCK inhibitor, hydroxyfasudil monohydrochloride, which has been shown to cross the blood-brain-barrier and reduce ischemia-induced brain damage [26], was used to further confirm that ROCK inhibition could be beneficial to axonal development in npc1−/− mice in vivo. Continuous administration of hydroxyfasudil monohydrochloride for 21 days not only increased p-p53 immunoreactivity, but also increased the number of axonal neurofilaments, as revealed by staining with SMI-312 antibody, especially in corpus callosum and striatum (FIGS. 14, A and B). Furthermore, ROCK inhibition also significantly increased SMI-3,2-immunopositive areas (FIG. 14C).

### Example 6

### Inhibition of Protein Synthesis Blocked U18666A-Induced RhoA Up-Regulation and Growth Cone Collapse

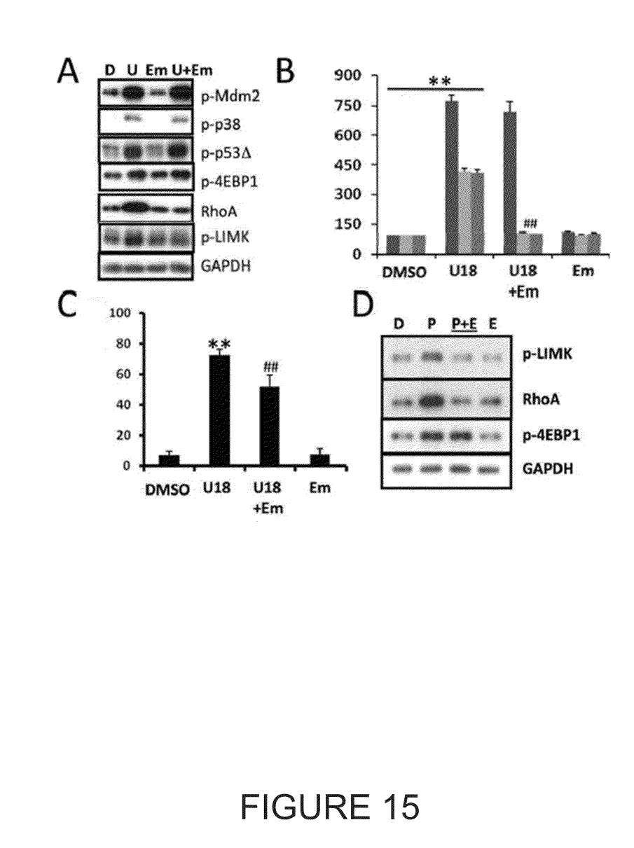

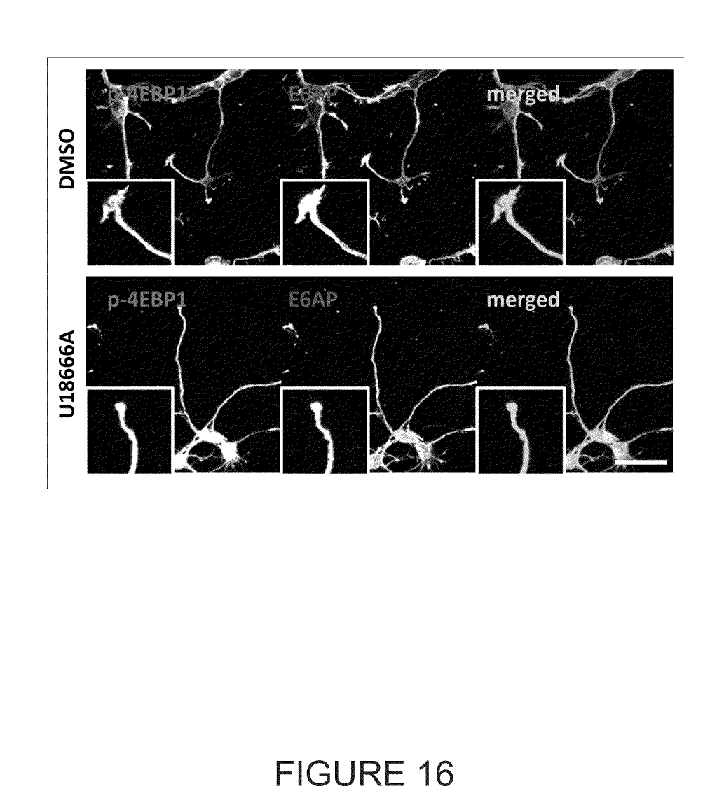

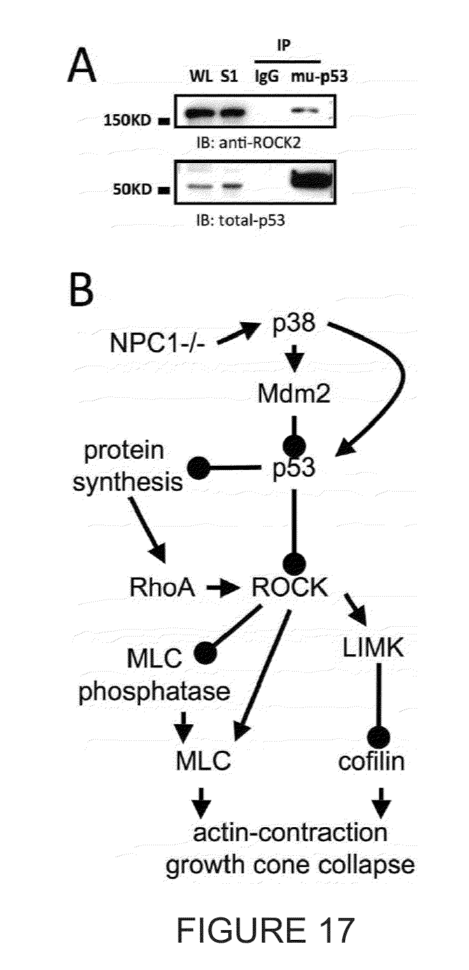

Emerging evidence indicates that rapid protein synthesis in axons and growth cones regulates growth cone behavior [27]. Wu et al [28] recently reported that RhoA transcripts are localized in developing axons and growth cones and that intra-axonal translation of the small GTPase is necessary and sufficient for semaphorin 3A-mediated growth cone collapse. Therefore, it was tested whether U18666A-induced growth cone collapse was associated with increased RhoA synthesis. U18666A treatment of cultured neurons from wild-type mice rapidly increased levels of phosphorylated 4EBP1 (p-4EBP1), a widely used marker of protein synthesis initiation (FIG. 15). Immunohistochemical studies confirmed that p-4EBP1 levels were increased in axons and growth cones (FIG. 16). Pre-treatment with emetine, a protein synthesis inhibitor, significantly reduced U18666A-induced increase in RhoA levels (FIG. 15). Emetine pretreatment also significantly reduced U18666A-induced phosphorylation of LIMK and growth cone collapse, suggesting that local RhoA synthesis may contribute to ROCK-dependent growth cone collapse (FIG. 15). Emetine treatment did not affect U18666A-induced changes in levels of p-Mdm2, p-p38, and p-p530 (FIG. 15A), indicating that RhoA protein synthesis is a downstream event. To further test the idea that p53 could interfere with ROCK signaling by suppressing RhoA synthesis, wild-type cortical neurons were treated with the p53 inhibitor pifithrin-μ in the presence or absence of emetine pre-treatment. Immunoblot results indicated that p53 inhibition induced a rapid increase in levels of RhoA and p-LIMK. Both events were blocked by emetine pretreatment (FIG. 15D). P53 inhibition also increased levels of p-4EBP1, further supporting the notion that p53 tonically inhibits protein synthesis. Immunoprecipitation experiments revealed a direct association between p53 and ROCK2, the most expressed isoform of ROCK in brain (FIG. 17A).

### REFERENCES

1. Kwon H J, Abi-Mosleh L, Wang M L, Deisenhofer J, Goldstein J L, et al. (2009) Structure of N-terminal domain of NPC1 reveals distinct subdomains for binding and transfer of cholesterol. Cell 137: 1213-1224.
2. Walkley S U, Suzuki K (2004) Consequences of NPC1 and NPC2 loss of function in mammalian neurons. Biochim Biophys Acta 1685: 48-62.
3. Baudry M, Yao Y, Simmons D, Liu J, Bi X (2003) Postnatal development of inflammation in a murine model of Niemann-Pick type C disease: immunohistochemical observations of microglia and astroglia. Exp Neurol 184: 887-903.
4. Higashi Y, Murayama S, Pentchev P G, Suzuki K (1993) Cerebellar degeneration in the Niemann-Pick type C mouse. Acta Neuropathol 85:175-184.
5. March P A, Thrall M A, Brown D E, Mitchell T W, Lowenthal A C, et al. (1997) GABAergic neuroaxonal dystrophy and other cytopathological alterations in feline Niemann-Pick disease type C. Acta Neuropathol 94: 164-172.
6. Zervas M, Somers K L, Thrall M A, Walkley SU (2001) Critical role for glycosphingolipids in Niemann-Pick disease type C. Curr Biol 11: 1283-1287.
7. Liao G, Yao Y, Liu J, Yu Z, Cheung 5, et al. (2007) Cholesterol accumulation is associated with lysosomal dysfunction and autophagic stress in Npc1−/− mouse brain. Am J Pathol 171: 962-975.
8. Auer I A, Schmidt M L, Lee V M, Curry B, Suzuki K, et al. (1995) Paired helical filament tau (PHFtau) in Niemann-Pick type C disease is similar to PHFtau in Alzheimer's disease. Acta Neuropathol 90: 547-551.
9. Suzuki K, Parker C C, Pentchev P G, Katz D, Ghetti B, et al. (1995) Neurofibrillary tangles in Niemann-Pick disease type C. Acta Neuropathol 89: 227-238.
10. Distl R, Treiber-Held S, Albert F, Meske V, Harzer K, et al. (2003) Cholesterol storage and tau pathology in Niemann-Pick type C disease in the brain. J Pathol 200: 104-111.
11. Saito Y, Suzuki K, Nanba E, Yamamoto T, Ohno K, et al. (2002) Niemann-Pick type C disease: accelerated neurofibrillary tangle formation and amyloid beta deposition associated with apolipoprotein E epsilon 4 homozygosity. Ann Neurol 52: 351-355.
12. Ong W Y, Kumar U, Switzer R C, Sidhu A, Suresh G, et al. (2001) Neurodegeneration in Niemann-Pick type C disease mice. Exp Brain Res 141:218-231.
13. Karten B, Vance D E, Campenot R B, Vance J E (2002) Cholesterol accumulates in cell bodies, but is decreased in distal axons, of Niemann-Pick C1-deficient neurons. J Neurochem 83: 1154-1163.
14. Tashiro Y, Yamazaki T, Shimada Y, Ohno-Iwashita Y, Okamoto K (2004) Axon-dominant localization of cell-surface cholesterol in cultured hippocampal neurons and its disappearance in Niemann-Pick type C model cells. Eur J Neurosci 20: 2015-2021.
15. de Chaves El, Rusinol A E, Vance D E, Campenot R B, Vance J E (1997) Role of lipoproteins in the delivery of lipids to axons during axonal regeneration. J Biol Chem 272: 30766-30773.
16. Guirland C, Suzuki S, Kojima M, Lu B, Zheng J Q (2004) Lipid rafts mediate chemotropic guidance of nerve growth cones. Neuron 42: 51-62.
17. Qin Q, Baudry M, Liao G, Noniyev A, Galeano J, et al. (2009) A novel function for p53: regulation of growth cone motility through interaction with Rho kinase. J Neurosci 29: 5183-5192.
18. Ishibashi S, Yamazaki T, Okamoto K (2009) Association of autophagy with cholesterol-accumulated compartments in Niemann-Pick disease type C cells. J Clin Neurosci 16: 954-959.
19. Bornig H, Geyer G (1974) Staining of cholesterol with the fluorescent antibiotic “filipin”. Acta Histochem 50: 110-115.
20. Kyriakis J M, Avruch J (2001) Mammalian mitogen-activated protein kinase signal transduction pathways activated by stress and inflammation. Physiol Rev 81: 807-869.
21. Heron-Milhavet L, LeRoith D (2002) Insulin-like growth factor I induces MDM2-dependent degradation of p53 via the p38 MAPK pathway in response to DNA damage. J Biol Chem 277: 15600-15606.
22. Jackson M W, Patt L E, LaRusch G A, Donner D B, Stark G R, et al. (2006) Hdm2 nuclear export, regulated by insulin-like growth factor-I/MAPK/p90Rsk signaling, mediates the transformation of human cells. Biol Chem 281:16814-16820.
23. Diskin R, Lebendiker M, Engelberg D, Livnah O (2007) Structures of p38alpha active mutants reveal conformational changes in L16 loop that induce autophosphorylation and activation. J Mol Biol 365: 66-76.
24. Hackeng C M, Relou I A, Pladet M W, Goiter G, van Rijn H J, et al. (1999) Early platelet activation by low density lipoprotein via p38MAP kinase. Thromb Haemost 82: 1749-1756.
25. Ishizaki T, Uehata M, Tamechika I, Keel I, Nonomura K, et al. (2000) Pharmacological properties of Y-27632, a specific inhibitor of rho-associated kinases. Mol Pharmacol 57: 976-983.
26. Satoh S, Toshima Y, Ikegaki I, Iwasaki M, Asano T (2007) Wide therapeutic time window for fasudil neuroprotection against ischemia-induced delayed neuronal death in gerbils. Brain Res 1128: 175-180.
27. Lin A C, Holt C E (2007) Local translation and directional steering in axons. EMBO J. 26: 3729-3736.
28. Wu K Y, Hengst U, Cox U, Macosko E Z, Jeromin A, et al. (2005) Local translation of RhoA regulates growth cone collapse. Nature 436: 1020-1024.
29. Haupt S, Louria-Hayon I, Haupt Y (2003) P53 licensed to kill? Operating the assassin. J Cell Biochem 88: 76-82.
30. Kubbutat M H, Jones S N, Vousden K H (1997) Regulation of p53 stability by Mdm2. Nature 387: 299-303.
31. Fang S, Jensen J P, Ludwig R L, Vousden K H, Weissman A M (2000) Mdm2 is a RING finger-dependent ubiquitin protein ligase for itself and p53. J Biol Chem 275: 8945-8951.
32. Grossman S R, Perez M, Kung A L, Joseph M, Mansur C, et al. (1998) p300/MDM2 complexes participate in MDM2-mediated p53 degradation. Mol Cell 2:405-415.
33. Brooks C L, Gu W (2006) p53 ubiquitination: Mdm2 and beyond. Mol Cell 21:307-315.
34. Lau L, Hansford L M, Cheng L S, Hang M, Baruchel S, et al. (2007) Cyclooxygenase inhibitors modulate the p53/HDM2 pathway and enhance chemotherapy-induced apoptosis in neuroblastoma. Oncogene 26: 1920-1931.
35. Zhou B P, Liao Y, Xia W, Zou Y, Spohn B, et al. (2001) HER-2/neu induces p53 ubiquitination via Akt-mediated MDM2 phosphorylation. Nat Cell Biol 3:973-982.
36. Mayo L D, Donner D B (2001) A phosphatidylinositol 3-kinase/Akt pathway promotes translocation of Mdm2 from the cytoplasm to the nucleus. Proc Natl Acad Sci USA 98: 11598-11603.
37. Wood N T, Meek D W, Mackintosh C (2009) 14-3-3 Binding to Pimphosphorylated Ser166 and Ser186 of human Mdm2-Potential interplay with the PKB/Akt pathway and p14(ARF). FEBS Lett 583: 615-620.
38. Campbell D S, Holt C E (2003) Apoptotic pathway and MAPKs differentially regulate chemotropic responses of retinal growth cones. Neuron 37: 939-952.
39. Yang S R, Kim S J, Byun K H, Hutchinson B, Lee B H, et al. (2006) NPC1 gene deficiency leads to lack of neural stem cell self-renewal and abnormal differentiation through activation of p38 mitogen-activated protein kinase signaling. Stem Cells 24: 292-298.
40. Olsson A, Manzi C, Strasser A, Villunger A (2007) How important are posttranslational modifications in p53 for selectivity in target-gene transcription and tumour suppression? Cell Death Differ 14: 1561-1575.
41. Bi X, Liu J, Yao Y, Baudry M, Lynch G (2005) Deregulation of the phosphatidylinositol-3 kinase signaling cascade is associated with neurodegeneration in Npc1−/− mouse brain. Am J Pathol 167: 1081-1092.
42. Mueller B K, Mack H, Teusch N (2005) Rho kinase, a promising drug target for neurological disorders. Nat Rev Drug Discov 4: 387-398.
43. Amano M, Chihara K, Nakamura N, Kaneko T, Matsuura Y, et al. (1999) The COOH terminus of Rho-kinase negatively regulates rho-kinase activity. J Biol Chem 274: 32418-32424.
44. Fu X, Gong M C, Jia T, Somlyo A V, Somlyo A P (1998) The effects of the Rho kinase inhibitor Y-27632 on arachidonic acid-, GTPgammaS-, and phorbol ester-induced Ca2+-sensitization of smooth muscle. FEBS Lett 440: 183-187.
45. Shirao S, Kashiwagi S, Sato M, Miwa S, Nakao F, et al. (2002) Sphingosylphosphorylcholine is a novel messenger for Rho-kinase-mediated Ca2+ sensitization in the bovine cerebral artery: unimportant role for protein kinase C. Circ Res 91:112-119.
46. Horton L E, Bushell M, Barth-Baus D, Tilleray V J, Clemens M J, et al. (2002) p53 activation results in rapid dephosphorylation of the eIF4E-binding protein 4E-BP1, inhibition of ribosomal protein S6 kinase and inhibition of translation initiation. Oncogene 21: 5325-5334.

The claimed invention is:

1. A method of treating a neurodegenerative disease, comprising administering a formulation to at least one central nervous system component of a mammal, wherein the formulation comprises an inhibitor of Rho-associated protein kinase (ROCK), and wherein the dosage of the ROCK inhibitor is sufficient to inhibit axonal degradation or growth cone collapse of a neuron.

2. The method of claim 1, wherein the inhibitor of ROCK inhibits ROCK that was activated as a consequence of degradation of the p53 tumor suppressor.

3. The method of claim 1, wherein the ROCK inhibitor is Y-27632 (trans-4-[(1R)-1-Aminoethyl]-N-4-pyridinylcyclohexanecarboxamide dihydrochloride), H 1152 ((S)-(+)-2-Methyl-1-[(4-methyl-5-isoquinolinyl)sulfonyl]-hexahydro-1H-1,4-diazepine dihydrochloride), or Fasudil Hydrochloride.

4. The method of claim 1, wherein the neurodegenerative disease is Alzheimer's disease, amyotrophic lateral sclerosis, Parkinson's disease, or Niemann-Pick type C disease.

5. The method of claim 1, wherein the axonal degradation or growth cone collapse of a neuron is caused in part or in whole by cholesterol deficiency in the axons.

6. The method of claim 5, wherein the cholesterol deficiency is caused by disregulation of intracellular cholesterol transport in neurons of at least one central nervous system component.

7. The method of claim 6, wherein the inhibition of cholesterol transport is due in part or in whole to a mutation in Niemann-Pick type C-1 (NPC1), NPC2, or both NPC1 and NPC2.

8. The method of claim 7, wherein the mutation of either NPC1 or NPC2 causes p53 degradation.

9. The method of claim 7, wherein the degradation of p53 is mediated by phosphorylation of Mdm2 by p38 MAPK.

10. A method of identifying at least one compound that can be used to treat a neurodegenerative disorder or disease, wherein the method comprises
(a) obtaining primary cultured mammalian neurons,
(b) dividing the neurons into at least four subcultures,
(c) pretreating at least f the subcultures by contacting the subcultured neurons with a solution of the test compound,
(d) pretreating at least two of the subcultures by contacting the subcultured neurons with a first control solution,
(e) treating at least one of the subcultures that was pretreated with the test compound, and at least one of the subcultures that was treated with the first control solution by contacting the cultured neurons with an agent that can trigger axon growth cone collapse, and
(f) determining whether the test compound inhibits axon growth cone collapse based on whether pretreatment of neurons with the active agent solution causes a reduction in the number of neurons that undergo growth cone collapse.

11. The method of claim 10, wherein the agent that can trigger axon growth cone collapse is 3b-[2-(diethylamino)ethoxy]-androst-5-en-17-one, monohydrochloride (U-18666A).

12. A method of identifying at least one compound that can be used to treat a neurodegenerative disorder or disease, wherein the method comprises
(a) obtaining primary cultured neurons from npc1−/− embryos and npc1+/+ control embryos,
(b) dividing the npc1−/− neurons into at least two subcultures,
(c) dividing the npc1+/+ neurons into at least two subcultures,
(d) pretreating at least one of the npc1−/− subcultures, and at least one of the npc1+/+ subcultures with a solution of the test compound by contacting the subcultured neurons with a solution of the test compound,
(e) pretreating at least one of the npc1−/− subcultures, and at least one of the npc1+/+ subcultures with a solution of a control solution by contacting the subcultured neurons with a control solution, and
(f) determining whether the test compound inhibits axon growth cone collapse in npc1−/− neuron cultures as compared to control solution-treated npc1−/− neuron cultures.
